SEQUENCE LISTING (48 sequences)

SEQ ID NO: 1 - protein, 49 aa, Artificial Sequence
  minimal consensus N-term
  VARIANT 31: any amino acid
  VARIANT 37: any amino acid
  VARIANT 39: any amino acid
PSVFLFPPKPKDTLMISRTPEVTCVVVDVSXEDPEVXFXWYVDGVEVHN

SEQ ID NO: 2 - protein, 40 aa, Artificial Sequence
  minimal consensus C-term
  VARIANT 4: any amino acid
  VARIANT 13: any amino acid
  VARIANT 31: any amino acid
  VARIANT 34: any amino acid
  VARIANT 35: any amino acid
NSTXRVVSVLTVXHQDWLNGKEYKCKVSNKXLPXXIEKTI

SEQ ID NO: 3 - protein, 49 aa, Artificial Sequence
  minimal N-term IgG1
PSVFLFPPKPKDTLMISRTPEVTCVVVDVSHEDPEVQFNWYVDGVEVHN

SEQ ID NO: 4 - protein, 40 aa, Artificial Sequence
  minimal C-term IgG1
NSTFRVVSVLTVVHQDWLNGKEYKCKVSNKGLPAPIEKTI

SEQ ID NO: 5 - protein, 49 aa, Artificial Sequence
  minimal N-term IgG2
PSVFLFPPKPKDTLMISRTPEVTCVVVDVSQEDPEVQFNWYVDGVEVHN

SEQ ID NO: 6 - protein, 40 aa, Artificial Sequence
  minimal C-term IgG2
NSTYRVVSVLTVLHQDWLNGKEYKCKVSNKGLPSSIEKTI

SEQ ID NO: 7 - protein, 49 aa, Artificial Sequence
  minimal N-term IgG3
PSVFLFPPKPKDTLMISRTPEVTCVVVDVSHEDPEVKFNWYVDGVEVHN

SEQ ID NO: 8 - protein, 40 aa, Artificial Sequence
  minimal C-term IgG3
NSTYRVVSVLTVLHQDWLNGKEYKCKVSNKALPAPIEKTI

SEQ ID NO: 9 - protein, 48 aa, Artificial Sequence
  minimal N-term IgG4
SVFLFPPKPKDTLMISRTPEVTCVVVDVSHEDPEVQFKWYVDGVEVHN

SEQ ID NO: 10 - protein, 40 aa, Artificial Sequence
  minimal C-term IgG4
NSTFRVVSVLTVLHQDWLNGKEYKCKVSNKALPAPIEKTI

SEQ ID NO: 11 - protein, 293 aa, Artificial Sequence
  IL13VL_GGGSGGGG_IL4VH_CH2(N)
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGSGGGGQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSGGPSVFLFPPKPKDTLMISRTPEVTCVVVDVSHEDPEVKFNWYVDGVEVHN

SEQ ID NO: 12 - protein, 308 aa, Artificial Sequence
  IL4VL_GGGSGGGG_IL13VH_GGGGSGGGGS_CH2(C)_[His6]
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSAKTKPREEQYNSTYRVVSVLTVLHQDWLNGKEYKCKVSNKALPAPIEKTISKAKGQPREHHHHHH

SEQ ID NO: 13 - protein, 386 aa, Artificial Sequence
  IL13VL_GGGGSGGGGS_IL4VL_IGKC_CH2(N)
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGGSGGGGSDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPSVFLFPPKPKDTLMISRTPEVTCVVVDVSHEDPEVKFNWYVDGVEVHN

SEQ ID NO: 14 - protein, 424 aa, Artificial Sequence
  IL13VH_GGGGSGGGGS_IL4VH_CH1_GGG_CH2(C)_[His]6
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGAKTKPREEQYNSTYRVVSVLTVLHQDWLNGKEYKCKVSNKALPAPIEKTISKAKGQPREHHHHHH

SEQ ID NO: 15 - protein, 391 aa, Artificial Sequence
  IL13VL_GQPKAAPS_IL4VL_TKGPSVF_IGKC_CH2(N)
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGQPKAAPSDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKTKGPSVFRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPSVFLFPPKPKDTLMISRTPEVTCVVVDVSHEDPEVKFNWYVDGVEVHN

SEQ ID NO: 16 - protein, 415 aa, Artificial Sequence
  IL4VH_S_IL13VH_CH1_GGG_CH2(C)_[His]6
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGAKTKPREEQYNSTYRVVSVLTVLHQDWLNGKEYKCKVSNKALPAPIEKTISKAKGQPREHHHHHH

SEQ ID NO: 17 - protein, 284 aa, Artificial Sequence
  IL13VL_CH2(N)_GGGGSGGGGSGGGGS_IL4VL
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGPSVFLFPPKPKDTLMISRTPEVTCVVVDVSHEDPEVKFNWYVDGVEVHNGGGGSGGGGSGGGGSDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIK

SEQ ID NO: 18 - protein, 321 aa, Artificial Sequence
  IL13VH_GGGGSGGGGSGGGGS_CH2(C)_IL4VH_[His]6
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGGGGSAKTKPREEQYNSTYRVVSVLTVLHQDWLNGKEYKCKVSNKALPAPIEKTISKAKGQPREQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSHHHHHH

SEQ ID NO: 19 - protein, 269 aa, Artificial Sequence
  IL13VL_CH2(N)_IL4VL
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGPSVFLFPPKPKDTLMISRTPEVTCVVVDVSHEDPEVKFNWYVDGVEVHNDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIK

SEQ ID NO: 20 - protein, 309 aa, Artificial Sequence
  IL13VH_GGG_CH2(C)_IL4VH_[His]6
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGAKTKPREEQYNSTYRVVSVLTVLHQDWLNGKEYKCKVSNKALPAPIEKTISKAKGQPREQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSHHHHHH

SEQ ID NO: 21 - protein, 537 aa, Artificial Sequence
  IL4VL_GGGSGGGG_IL13VH_CH2(C)_GGGSGSA_IgG4- Fc(PE)
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSAKTKPREEQYNSTYRVVSVLTVLHQDWLNGKEYKCKVSNKALPAPIEKTISKAKGQPREGGGSGSAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 653 aa, Artificial Sequence
  IL13VH_GGGGSGGGGS_IL4VH_CH1_GGG_CH2(C)_ GGGSGSA_IgG4-Fc(PE)
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGAKTKPREEQYNSTYRVVSVLTVLHQDWLNGKEYKCKVSNKALPAPIEKTISKAKGQPREGGGSGSAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 644 aa, Artificial Sequence
  IL4VH_S_IL13VH_CH1_GGG_CH2(C)_GGGSGSA_IgG4- Fc(PE)
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGAKTKPREEQYNSTYRVVSVLTVLHQDWLNGKEYKCKVSNKALPAPIEKTISKAKGQPREGGGSGSAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 444 aa, Artificial Sequence
  IL13_IgG4-Fc(PE)_holes_RF
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 25 - protein, 160 aa, Artificial Sequence
  IL4VL_GG_CH2(N)
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGGPSVFLFPPKPKDTLMISRTPEVTCVVVDVSHEDPEVKFNWYVDGVEVHN

SEQ ID NO: 26 - protein, 429 aa, Artificial Sequence
  IL4VH_GGGGGSGGGGGS_CH2(C)_GGGSGSA_IgG4-Fc(PE)_ knobs
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSGGGGGSGGGGGSAKTKPREEQYNSTYRVVSVLTVLHQDWLNGKEYKCKVSNKALPAPIEKTISKAKGQPREGGGSGSAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 218 aa, Artificial Sequence
  Il13LC
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 413 aa, Artificial Sequence
  IL4VL_GGGGGSGGGGGS_CH2(C)_GGGSGSA_ IgG4-Fc(PE)_ knobs
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGGGSGGGGGSAKTKPREEQYNSTYRVVSVLTVLHQDWLNGKEYKCKVSNKALPAPIEKTISKAKGQPREGGGSGSAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 176 aa, Artificial Sequence
  IL4VH_GG_CH2(N)
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSGGGGPSVFLFPPKPKDTLMISRTPEVTCVVVDVSHEDPEVKFNWYVDGVEVHN

SEQ ID NO: 30 - protein, 51 aa, Artificial Sequence
  N-term
GGPSVFLFPPKPKDTLMISRTPEVTCVVVDVSHEDPEVKFNWYVDGVEVHN

SEQ ID NO: 31 - protein, 59 aa, Artificial Sequence
  C-term
AKTKPREEQYNSTYRVVSVLTVLHQDWLNGKEYKCKVSNKALPAPIEKTISKAKGQPRE

SEQ ID NO: 32 - protein, 19 aa, Artificial Sequence
  Leader Sequence (used in all constructs) Aa 1-19 from Mouse Ig heavy chain V region 102 (UniProt P01750)
MGWSCIILFLVATATGVHS

SEQ ID NO: 33 - protein, 116 aa, Artificial Sequence
  CH2_[His]6
GGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREHHHHHH

SEQ ID NO: 34 - protein, 4 aa, Artificial Sequence
  Linker
  VARIANT 1: w-fold (0-20)
  VARIANT 1..3: z-fold (0-10)
  VARIANT 2: x-fold (0-10)
  VARIANT 3: y-fold (0-20)
  VARIANT 4: missing (only to satisfy minimum length “4” for seq listing, see features for other residues)
GSGX

SEQ ID NO: 35 - protein, 12 aa, Artificial Sequence
  Linker
GGGGGSGGGGGS

SEQ ID NO: 36 - protein, 15 aa, Artificial Sequence
  Linker
GGGGSGGGGSGGGGS

SEQ ID NO: 37 - protein, 8 aa, Artificial Sequence
  Linker
GGGSGGGG

SEQ ID NO: 38 - protein, 10 aa, Artificial Sequence
  Linker
GGGGSGGGGS

SEQ ID NO: 39 - protein, 8 aa, Artificial Sequence
  Linker
GQPKAAPS

SEQ ID NO: 40 - protein, 6 aa, Artificial Sequence
  Linker
TKGPSV

SEQ ID NO: 41 - protein, 288 aa, Artificial Sequence
  IL13VL_GGGSGGGG_IL4VH_GG_IgA-CH2(N)122-166
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGSGGGGQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSGGCHPRLSLHRPALEDLLLGSEANLTCTLTGLRDASGVTFTWTPSS

SEQ ID NO: 42 - protein, 308 aa, Artificial Sequence
  IL4VL_GGGSGGGG_IL13VH_GGGGSGGGGS_IgA-CH2(C)167- 225_C180S_[His6]
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGKSAVQGPPERDLSGCYSVSSVLPGCAEPWNHGKTFTCTAAYPESKTPLTATLSKSGNTHHHHHH

SEQ ID NO: 43 - protein, 290 aa, Artificial Sequence
  IL13VL_GGGSGGGG_IL4VH_IgD-CH2(N)166-213
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGSGGGGQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSQPLGVYLLTPAVQDLWLRDKATFTCFVVGSDLKDAHLTWEVAGKVPTG

SEQ ID NO: 44 - protein, 310 aa, Artificial Sequence
  IL4VL_GGGSGGGG_IL13VH_GGGGSGGGGS_IgD-CH2(C)214- 274_[His6]
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGVEEGLLERHSNGSQSQHSRLTLPRSLWNAGTSVTCTLNHPSLPPQRLMALREPAAQAPVKHHHHHH

SEQ ID NO: 45 - protein, 291 aa, Artificial Sequence
  IL13VL_GGGSGGGG_IL4VH_IgE-CH3(N)215-263
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGSGGGGQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSRGVSAYLSRPSPFDLFIRKSPTITCLVVDLAPSKGTVNLTWSRASGKPV

SEQ ID NO: 46 - protein, 308 aa, Artificial Sequence
  IL4VL_GGGSGGGG_IL13VH_GGGGSGGGGS_IgE-CH3(C)264- 322_[His6]
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSNHSTRKEEKQRNGTLTVTSTLPVGTRDWIEGETYQCRVTHPHLPRALMRSTTKTSGPRAHHHHHH

SEQ ID NO: 47 - protein, 289 aa, Artificial Sequence
  IL13VL_GGGSGGGG_IL4VH_IgE-CH2(N)110-156(C121S)
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGGSGGGGQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSTPPTVKILQSSSDGGGHFPPTIQLLCLVSGYTPGTINITWLEDGQVM

SEQ ID NO: 48 - protein, 301 aa, Artificial Sequence
  IL4VL_GGGSGGGG_IL13VH_GGGGSGGGGS_IgE-CH2(C)157- 208_[His6]
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGGSGGGGEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSDVDLSTASTTQEGELASTQSELTLSQKHWLSDRTYTCQVTYQGHTFEDSTKKHHHHHH

(19) United States
(12) Patent Application Publication
(10) Pub. No.: US 2023/0103563 A1
(43) Pub. Date: Apr. 6, 2023

(54) SPLIT CH2 DOMAINS

(71) Applicant: SANOFI, Paris (FR)

(72) Inventors: Werner DITTRICH, Frankfurt am Main (DE); Ingo FOCKEN, Frankfurt am Main (DE); Christian LANGE, Frankfurt am Main (DE); Thomas LANGER, Frankfurt am Main (DE); Ercole RAO, Frankfurt am Main (DE)

(21) Appl. No.: 17/908,190

(22) PCT Filed: Mar. 29, 2021
(86) PCT No.: PCT/EP2021/058200

§ 371 Date: Aug. 30, 2022

(30) Foreign Application Priority Data
Mar. 30, 2020 (EP) 20315072.7

(51) Int. Cl. C07K 16/24

(52) U.S. Cl. CPC C07K 16/247; C07K 16/244; C07K 2317/626; C07K 2317/94; C07K 2317/92; A61K 39/00

(57) ABSTRACT

The invention relates to a protein complex comprising at least two polypeptide chains A (PCA) and B (PCB), wherein PCA comprises a heterodimerization domain A (HDA) and PCB comprises a heterodimerization domain B (HDB), wherein HDA and HDB bind to each other and wherein one heterodimerization domain comprises or consists of two N-terminal β-strands (N-β) of an immunoglobulin (Ig) domain and the other heterodimerization domain comprises or consists of two C-terminal β-strands (C-β) of an Ig domain. The invention further relates to polynucleotides encoding one or more polypeptides of the protein complex, expression vectors comprising the polynucleotides and a cell comprising the polynucleotides or the expression vectors.

The present invention relates to the field of protein heterodimerization mediated by specialized heterodimerization domains. In particular, the invention relates to a protein complex comprising at least two polypeptide chains with heterodimerization domains that bind to each other. One heterodimerization domain comprises N-terminal β-strands of an immunoglobulin (Ig) domain and the other heterodimerization domain comprises C-terminal β-strands of an Ig domain.

### BACKGROUND OF THE INVENTION

The phenomenon of protein complementation was first described more than 50 years ago when Ullman et al. discovered that a peptide is able to restore the activity of β-galactosidase (Ullmann et al.; J Mol Biol. 1965; 12:918-923). Since this seminal work numerous examples of protein complementation have been described. The underlying principle is that a protein is split at a distinct site and the two parts are able to re-fold into a functional protein when brought together. The proteins used in this approach are usually enzymes (e.g. dihydrofolate-reductase, glycinamide ribonucleotide transformylase, aminoglycoside phosphotransferase, β-lactamase or luciferase) but can also be non-enzymatic proteins like ubiquitin and fluorescent proteins. Protein complementation techniques are often used to study protein-protein interactions. However, the phenomenon of protein complementation can also be used for other applications.

Monoclonal antibodies are very promising candidates for new treatment options, in particular for the treatment of cancer. In contrast to natural antibodies which are bivalent but monospecific (i.e. they recognize only one target), bispecific antibodies are able to bind two different targets or epitopes simultaneously. Multispecific antibodies can be grouped into several classes. In one example, bispecific antibodies consisting only of antigen-binding domains lack the Fc domain. These bispecific antibodies are small, may be produced in microbial systems, but do not evoke antibody-dependent cell-mediated cytotoxicity (ADCC) or complement-dependent cytotoxicity (CDC) due to the absence of the Fc domain. The Fc-domain mediates these effects by binding of to the Fc-receptors and the complement C1q protein, respectively. The Fc domain is further responsible for the long plasma half-life of antibodies. This is caused by binding of the Fc domain to the receptor FcRn, which enables recycling of the antibody during circulation. If the Fc domain is absent, much shorter plasma half-lives are obtained. In another example, heterodimeric bispecific antibodies may have two different heavy chains and two corresponding light chains. They can be generated by coexpression of two expression cassettes each comprising a heavy chain and a light chain. However, this approach leads to the formation of many unwanted mispaired side products. The heavy chains form homodimers as well as the desired heterodimers (the “heavy chain pairing problem”). Several methods have been developed to achieve heterodimerization of the heavy chains such as the introduction of the so called knob-into-holes-mutations or the introduction of electrostatic steering mutations. But with heterodimerized heavy chains still one problem remains: the “light chain pairing problem”, which relates to the correct pairing of two different light chains to their corresponding heavy chains. One possibility is to use a common light chain. However, this approach is not feasible in all cases.

The present invention addresses the mispairing problem and achieves the desired pairing of different chains of multispecific antibodies in the same cell with minimal or even without any undesired by-products, i.e. chain pairings other than the desired ones. This is achieved by using novel heterodimerization domains derived from a split immunoglobulin (Ig) domain.

### SUMMARY OF THE INVENTION

In a first aspect, the present invention relates to a protein complex comprising at least two polypeptide chains A (PCA) and B (PCB), wherein PCA comprises a heterodimerization domain A (HDA) and PCB comprises a heterodimerization domain B (HDB), wherein HDA and HDB bind to each other and wherein one heterodimerization domain comprises or consists of two N-terminal β-strands (N-β) of an immunoglobulin (Ig) domain and the other heterodimerization domain comprises or consists of two C-terminal β-strands (C-β) of an Ig domain.

In a second aspect, the present invention relates to one or more polynucleotides encoding one or more polypeptides of the protein complex according to the first aspect of the invention.

In a third aspect, the present invention relates to one or more expression vectors comprising the one or more polynucleotides according to the second aspect of the invention.

In a fourth aspect, the present invention relates to a cell comprising the one or more polynucleotides according to the second aspect of the invention or the one more expression vectors according to the third aspect of the invention.

In a fifth aspect, the present invention relates to a pharmaceutical composition comprising a pharmaceutically acceptable carrier and the protein complex according to the first aspect of the invention, the one or more polynucleotides according to the second aspect of the invention or the one or more expression vectors according to the third aspect of the invention.

### DETAILED DESCRIPTION OF EMBODIMENTS

Before the present invention is described in detail below, it is to be understood that this invention is not limited to the particular methodology, protocols and reagents described herein as these may vary. It is also to be understood that the terminology used herein is for the purpose of describing embodiments only, and is not intended to limit the scope of the present invention, which will be limited only by the appended claims. Unless defined otherwise, all technical and scientific terms used herein have the same meanings as commonly understood by one of ordinary skill in the art. Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

To practice the present invention, unless otherwise indicated, conventional methods of chemistry, biochemistry, and recombinant DNA techniques are employed which are explained in the literature in the field (cf, e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

In the description that follows, amino acid numbers are used with respect to antibodies, which do not refer to a SEQ ID NO. These numbers refer to amino acid positions in antibodies according to the UniProtKB database (www.uniprot.org/uniprot) in the version as disclosed on Aug. 26, 2016. Unless specified otherwise, the number(s) correspond to a position in human IgG, in particular in human IgG1. The UniProtKB sequences (in the version as disclosed on Aug. 26, 2016) of antibody domains referred to herein, including those of human IgG1 are incorporated by reference as embodiments of the domains described herein, as well as variants thereof as defined below.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments, which combine the explicitly described embodiments with any number of the disclosed and/or particular elements. Furthermore, any permutations and combinations of all described elements in this application should be considered disclosed by the description of the present application unless the context indicates otherwise.

In a first aspect, the present invention relates to a protein complex comprising at least two polypeptide chains A (PCA) and B (PCB), wherein PCA comprises a heterodimerization domain A (HDA) and PCB comprises a heterodimerization domain B (HDB), wherein HDA and HDB bind to each other and wherein one heterodimerization domain comprises or consists of two N-terminal β-strands of an immunoglobulin (Ig) domain (N-β) and the other heterodimerization domain comprises or consists of two C-terminal β-strands of an Ig domain (C-β).

In an embodiment of the present invention, one of HDA or HDB, which comprises two N-terminal β-strands, does not comprise one or more C-terminal β-strands (C-ß) and the other one, which comprises two C-terminal β-strands, does not comprise one or more N-terminal β-strands (N-ß). In other words, HDA and/or HDB may not be complete IgG domains consisting of C-terminal β-strands (C-β) and N-terminal β-strands (N-β).

In an embodiment of the present invention, HDA and HDB each have a length of 20-80 amino acids, in one embodiment HDA and HDB each have a length of 30-70 amino acids, in one embodiment HDA and HDB each have a length of 35-65 amino acids.

In the context of the present specification, the terms “immunoglobulin (Ig) domain” or “immunoglobulin fold” are used interchangeably to refer to a protein domain that consists of a 2-layer sandwich of 7-9 antiparallel β-strands arranged in two β-sheets with a Greek key topology. The Ig domain is probably the most frequently used “building block” in naturally occurring proteins. Proteins containing Ig domains are subsumed into the immunoglobulin superfamily. Not only antibodies, but also cell adhesion molecules, T-cell receptors, Fcγ-receptors and many more belong to this protein family. The immunoglobulin fold has been described thoroughly in a review article by Bork et al. (“The immunoglobulin fold. Structural classification, sequence patterns and common core”. September 1994; J. Mol. Biol. 242 (4): 309-20). Within the present specification, the nomenclature of the individual β-strands of an Ig domain (i.e. β-strands a, b, c, c′, c″, d, e, f and g) corresponds to the nomenclature used in Bork et al. The backbone characterized by the amino acid sequence of the Ig domain switches repeatedly between the two β-sheets. Thus, β-strand a may belong to the first or second sheet, β-strand b belongs to the first sheet, β-strand c belongs to the second sheet, β-strands c′ and c″ (if present) also belong to the second sheet, β-strand d (if present) again belongs to the first sheet, β-strand e belongs to the first sheet and β-strands f and g belong to the second sheet.

Of the 7-9 β-strands, strands a, b, c, c′ and c″ are considered N-terminal, wherein strands d, e, f and g are considered C-terminal.

In the context of the present specification, “N-β” is used to refer to an amino acid sequence comprising or consisting of the N-terminal β-strands of an Ig domain that are present within HDA or HDB. In instances where HDA (or HDB) comprises only two N-terminal β-strands of an Ig domain, N-β refers to an amino acid sequence comprising those two β-strands. In instances where HDA (or HDB) comprises three N-terminal β-strands of an Ig domain, N-β refers to an amino acid sequence comprising those three β-strands. HDA (or HDB) may comprise additional amino acids apart from those of N-β.

In the context of the present specification, “C-β” is used to refer to an amino acid sequence comprising or consisting of the C-terminal β-strands of an Ig domain that are present within HDA or HDB. In instances where HDB (or HDA) comprises only two C-terminal β-strands of an Ig domain, C-β refers to an amino acid sequence comprising those two β-strands. In instances where HDB (or HDA) comprises three to five C-terminal β-strands of an Ig domain, C-β refers to an amino acid sequence comprising those three to five β-strands. HDB (or HDA) may comprise additional amino acids apart from those of C-β.

Ig domains such as the CH2 domain of human IgG1 can be expressed in high amounts in prokaryotic systems, e.g. E. coli.

Surprisingly, the inventors have found that if the N-terminal part and the C-terminal part of an Ig domain are expressed as two separate polypeptides, the two parts are capable of reassembling into a complete Ig domain. Due to this property, the N- and C-terminal parts of a split Ig domain can be used as heterodimerization domains that enable the specific dimerization of two polypeptides fused to the N- and C-terminal part, respectively. An important application of such a heterodimerization domain is the controlled assembly of protein complexes, in particular multispecific antibodies, antibody derivatives or antibody-like molecules.

The term “antibody” as used herein refers to a molecule having the overall structure of an antibody, for example an IgG antibody. When referring to IgG in general, IgG1, IgG2, IgG3 and IgG4 are included, unless defined otherwise. IgG antibody molecules are Y-shaped molecules comprising four polypeptide chains: two heavy chains and two light chains. Each light chain consists of two domains, the N-terminal domain being known as the variable or VL domain (or region) and the C-terminal domain being known as the constant (or CL) domain (constant kappa (Cκ) or constant lambda (Cλ) domain). Each heavy chain consists of four domains. The N-terminal domain of the heavy chain is known as the variable (or VH) domain (or region), which is followed by the first constant domain (CH1), the hinge region, and then the second and third constant domains (CH2 and CH3). In an assembled antibody, the VL and VH domains associate to form an antigen binding site. Also, the CL and CH1 domains associate to keep one heavy chain associated with one light chain. The two heavy-light chain heterodimers associate by interaction of the CH2 and CH3 domains and interaction between the hinge regions of the two heavy chains. The term “antibody” as used herein also includes molecules which may have chimeric domain replacements (i.e. at least one domain replaced by a domain from a different antibody), such as an IgG1 antibody comprising an IgG3 domain (e.g. the CH3 domain of IgG3). Further, the term generally refers to multispecific, e.g. bispecific or trispecific antibodies.

The term “antibody derivative” as used herein refers to a molecule comprising at least the domains it is specified to comprise, but not having the overall structure of an antibody such as IgA, IgD, IgE, IgG, IgM, IgY or IgW, although still being capable of binding a target molecule. Said derivatives may be, but are not limited to functional (i.e. target binding, particularly specific target binding) antibody fragments or combinations thereof. It also relates to an antibody to which further antibody domains have been added, such as further variable domains. Thus, the term antibody derivative also includes multispecific (bispecific, trispecific, tetraspecific, pentaspecific hexaspecific etc.) and multivalent (bivalent, trivalent, tetravalent etc.) antibodies.

Bispecific antibodies occur in a plurality of formats (Brinkmann and Kontermann, Mabs 2017, Vol. 9, No. 2, 182-212). Examples for bispecific antibodies consisting only of antigen binding domains are bivalent Fabs (bi-Fabs), e.g. the DVD-Fabs or CODV-Fabs described herein. Another example are formats comprising only variable domains (Fv) but no constant domains, such as the “diabodies”, or “splite diabodies” described herein. Formats comprising only variable domains have the advantage of a very low molecular weight leading to a good tumor penetrance, which is important for oncologic applications. A disadvantage is however a low plasma half-life due to the lack of a constant domain, which mediates binding to the FcRn.

Homodimeric bispecific antibodies can be obtained by fusing a scFv to the heavy or light chain or by adding additional Fv domains to the heavy and light chains, respectively. Examples for these kinds of formats are the “dual variable domain” (DVD) configuration (which is also termed “tetravalent bispecific tandem immunoglobulin” (TBTI) configuration) and the cross-over dual variable” (CODV) configuration as described herein (Wu et al.; Nat Biotechnol. 2007; 25:1290-1297 and Steinmetz et al.; Mabs 2016; 8:867-87).

The term “antibody like-molecule” as used within the context of the present specification comprises antibody derivatives and antibody mimetics. The term “antibody mimetic” refers to compounds which can specifically bind antigens, similar to an antibody, but are not structurally related to antibodies. Usually, antibody mimetics are artificial peptides or proteins with a molar mass of about 3 to 20 kDa which comprise one, two or more exposed domains specifically binding to an antigen. Typically, such an antibody mimetic comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Examples include inter alia the LACI-D1 (lipoprotein-associated coagulation inhibitor); affilins, e.g. human-γ B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunitz domain of protease inhibitors; monobodies, e.g. the 10th type III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II; affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

The term “antigen” is used to refer to a substance, preferably an immunogenic peptide that comprises at least one epitope, preferably an epitope that elicits a B or T cell response or B cell and T cell response.

An “epitope”, also known as antigenic determinant, is that part of a substance, e.g. an immunogenic polypeptide, which is recognized by the immune system. Preferably, this recognition is mediated by the binding of antibodies, B cells, or T cells to the epitope in question. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. The term “epitope” comprises both conformational and non-conformational epitopes. Conformational and non-conformational epitopes are distinguished in that the binding to the former but not the latter is lost in the presence of denaturing solvents.

An immunogenic polypeptide according to the present invention can be derived from a pathogen. In some embodiments, the pathogen is selected from selected from the group consisting of viruses, bacteria and protozoa. However, in an alternative embodiment of the present invention the immunogenic polypeptide is a tumor antigen, i.e. polypeptide or fragment of a polypeptide specifically expressed by a cancer.

In one embodiment, N-β comprises or consists of a continuous amino acid sequence of an Ig domain comprising or consisting of β-strand b and c.

In one embodiment, N-β comprises or consists of a continuous amino acid sequence of an Ig domain comprising or consisting of β-strand a, b and c.

In one embodiment, C-β comprises or consists of a continuous amino acid sequence of an Ig domain comprising or consisting β-strand e and f.

In one embodiment, C-β comprises or consists of a continuous amino acid sequence of an Ig domain comprising or consisting of β-strand e, f and g.

In one embodiment, N-β comprises or consists of a continuous amino acid sequence of an Ig domain comprising or consisting of β-strand b and c and C-β comprises or consists of a continuous amino acid sequence of an Ig domain comprising or consisting of β-strand e and f.

In one embodiment, N-β comprises or consists of a continuous amino acid sequence of an Ig domain comprising or consisting of β-strand a to c and C-β comprises or consists of a continuous amino acid sequence of an Ig domain comprising or consisting of β-strand e to g.

In one embodiment, the Ig domains of N-β and C-β are independently selected from a heavy chain constant domain 2 (CH2) or a heavy chain constant domain 3 (CH3). In other words, N-β comprises or consists of an N-terminal amino acid sequence of a CH2 domain or CH3 domain and C-β comprises or consists of a C-terminal amino acid sequence of a CH2 domain or CH3 domain.

In one embodiment, the Ig domains of N-β and C-β are selected from the same CH2 domain or CH3 domain. In other words, N-β comprises or consists of an N-terminal amino acid sequence of a CH2 domain or CH3 domain and C-β comprises or consists of a C-terminal amino acid sequence of the same CH2 domain or CH3 domain.

In one embodiment, N-β comprises or consists of a continuous amino acid sequence of a CH2 domain or CH3 domain comprising or consisting of β-strand b and c.

In one embodiment, N-β comprises or consists of a continuous amino acid sequence of a CH2 domain or CH3 domain comprising or consisting of β-strand a to c.

In one embodiment, C-β comprises or consists of a continuous amino acid sequence of a CH2 domain or CH3 domain comprising or consisting of β-strand e and f.

In one embodiment, C-β comprises or consists of a continuous amino acid sequence of a CH2 domain or CH3 domain comprising or consisting of β-strand e to g.

In one embodiment, N-β comprises or consists of a continuous amino acid sequence of a CH2 domain or CH3 domain comprising or consisting of β-strand b and c and C-β consists of a continuous amino acid sequence of a CH2 domain or CH3 domain comprising or consisting of β-strand e and f.

In one embodiment, N-β comprises or consists of a continuous amino acid sequence of a CH2 domain or CH3 domain comprising or consisting of β-strand a to c and C-β comprises or consists of a continuous amino acid sequence of a CH2 domain or CH3 domain comprising or consisting of β-strand e to g.

In the context of the present specification, when referring to the CH2 domain in general, this includes the second constant domains present in the heavy chain of an IgA, IgD, IgE or IgG antibody. In particular, CH2 refers to a CH2 domain of IgG, particularly IgG1, IgG2, IgG3 or IgG4. CH2 domains have no protein-protein contacts to other domains within an antibody. CH2 domains comprise an intramolecular disulfide bond that stabilizes the tertiary structure of the domain. CH2 domains have the further advantage that they remain stable monomers if expressed in a bacterial expression system. In an IgM or IgE molecule, the CH3 domain corresponds to the CH2 domain of IgG, IgA, or IgD.

In one embodiment, the Ig domains of N-β and C-β are independently selected from an IgA, IgD, IgE or IgG heavy chain constant domain 2 (CH2) or an IgM or IgE heavy chain constant domain 3 (IgM CH3, IgE CH3). In other words, N-β comprises or consists of an N-terminal amino acid sequence of a CH2 domain or IgM or IgE CH3 domain and C-β comprises or consists of a C-terminal amino acid sequence of a CH2 domain or IgM or IgE CH3 domain.

In one embodiment, the Ig domains of N-β and C-β are selected from the same CH2 domain or IgM or IgE CH3 domain. In other words, N-β comprises or consists of an N-terminal amino acid sequence of a CH2 domain or IgM or IgE CH3 domain and C-β comprises or consists of a C-terminal amino acid sequence of the same CH2 domain or IgM or IgE CH3 domain.

In one embodiment, N-β comprises or consists of a continuous amino acid sequence of a CH2 domain or IgM or IgE CH3 domain comprising or consisting of β-strand b and c.

In one embodiment, N-β comprises or consists of a continuous amino acid sequence of a CH2 domain or IgM or IgE CH3 domain comprising or consisting of β-strand a to c.

In one embodiment, C-β comprises or consists of a continuous amino acid sequence of a CH2 domain or IgM or IgE CH3 domain comprising or consisting of β-strand e and f.

In one embodiment, C-β comprises or consists of a continuous amino acid sequence of a CH2 domain or IgM or IgE CH3 domain comprising or consisting of β-strand e to g.

In one embodiment, N-β comprises or consists of a continuous amino acid sequence of a CH2 domain or IgM or IgE CH3 domain comprising or consisting of β-strand b and c and C-β comprises or consists of a continuous amino acid sequence of a CH2 domain or IgM or IgE CH3 domain comprising or consisting of β-strand e and f.

In one embodiment, N-β comprises or consists of a continuous amino acid sequence of a CH2 domain or IgM or IgE CH3 domain comprising or consisting of β-strand a to c and C-β comprises or consists of a continuous amino acid sequence of a CH2 domain or IgM or IgE CH3 domain comprising or consisting of β-strand e to g.

In one embodiment, the Ig domains of N-β and C-β are IgG CH2 domains. In one embodiment, the Ig domains of N-β and C-β are independently selected from an IgG1, IgG2, IgG3, or IgG4 CH2 domain. In one embodiment, the Ig domains of N-β and C-β are selected from the same IgG CH2 domain.

In one embodiment, N-β comprises or consists of a continuous amino acid sequence of an IgG CH2 domain comprising or consisting of β-strand b and c.

In one embodiment, N-β comprises or consists of a continuous amino acid sequence of an IgG CH2 domain comprising or consisting of β-strand a to c.

In one embodiment, C-β comprises or consists of a continuous amino acid sequence of an IgG CH2 domain comprising or consisting of β-strand e and f.

In one embodiment, C-β comprises or consists of a continuous amino acid sequence of an IgG CH2 domain comprising or consisting of β-strand e to g.

In one embodiment, N-β comprises or consists of a continuous amino acid sequence of an IgG CH2 domain comprising or consisting of β-strand b and c and C-β comprises or consists of a continuous amino acid sequence of an IgG CH2 domain comprising or consisting of β-strand e and f.

In one embodiment, N-β comprises or consists of a continuous amino acid sequence of an IgG CH2 domain comprising or consisting of β-strand a to c and C-β comprises or consists of a continuous amino acid sequence of an IgG CH2 domain comprising or consisting of β-strand e to g.

In one embodiment, HDA and HDB (i) non-covalently or (ii) non-covalently and covalently bind to each other. In one embodiment, HDA and HDB non-covalently and covalently bind to each other.

In one embodiment, the covalent bond is an intermolecular disulfide bond. The native CH2 domain comprises an intramolecular disulphide bond. In instances where HDA and HDB are derived from a CH2 domain (i.e. where N-β comprises or consists of an N-terminal amino acid sequence of a CH2 domain and C-β comprises or consists of a C-terminal amino acid sequence of a CH2 domain), in some embodiments from the same CH2 domain, the intermolecular disulfide bond linking HDA and HDB can correspond to the intramolecular disulfide bond of the “parental” CH2 domain. The disulfide bond is a covalent bond. Thus, the disulphide bond ensures that the N- and C-terminal part of reassembled Ig domain are united into a single molecule. This is an advantage compared to known split protein approaches (protein complementation approaches). HDA and HDB may be bonded through one or more covalent bond, either parental and/or not parental.

In one embodiment, C-β comprises one or more Lys residues at its C-terminus. In one embodiment, C-β comprises 1, 2 or 3 Lys residues within its 10 most C-terminal amino acids. The Lys residues may be naturally occurring Lys residues that are present in the native Ig domain or may be non-naturally occurring Lys residues that have been introduced into the Ig domain. The C-terminal Lys residues are advantageous because they stabilize the tertiary structure of the reassembled Ig domain.

In one embodiment, C-β comprises or consists of a continuous amino acid sequence of a CH2 domain comprising or consisting of at least β-strand e and f and one or more Lys residues at its C-terminus. In one embodiment, C-β comprises or consists of a continuous amino acid sequence of an IgG CH2 domain comprising or consisting of at least β-strand e and f and one or more Lys residues at its C-terminus. In one embodiment, C-β comprises or consists of a continuous amino acid sequence of a CH2 domain comprising or consisting of at least β-strand d to g and optionally one or more Lys residues at its C-terminus. In one embodiment, C-β comprises or consists of a continuous amino acid sequence of an IgG CH2 domain comprising or consisting of at least β-strand d to g and optionally one or more Lys residues at its C-terminus. In one embodiment, C-β or consists of comprises a continuous amino acid sequence of a CH2 domain comprising or consisting of β-strands c′, d, e, f and g and optionally one or more Lys residues at its C-terminus. In one embodiment, C-β or consists of comprises a continuous amino acid sequence of an IgG CH2 domain comprising or consisting of β-strands c′, d, e, f and g and optionally one or more Lys residues at its C-terminus.

In one embodiment, the N-β and C-β each comprise a non-naturally occurring Cys residue and the Cys residues replace amino acids in the folded N-β and C-β, respectively, that naturally have a distance of between 3 to 7.5 Å between their Cα-atoms. In the reassembled Ig domain, the non-naturally occurring Cys residue in N-β and the non-naturally occurring Cys residue in C-β will have a distance that allows for the formation of a disulfide bond. A thus introduced disulfide bond is advantageous because it will stabilize the tertiary structure of the reassembled Ig domain.

In the context of the present invention, SEQ ID NO 1 relates to the amino acid sequence

PSVFLFPPKPKDTLMISRTPEVTCVVVDVSX₁
EDPEVX₂FX₃WYVDGVEVHN.

In the context of the present invention, SEQ ID NO 2 relates to the amino acid sequence

NSTX₄RVVSVLTVX₅HQDWLNGKEYKCKVSNKX₆LPX₇X₈IEKTI.

In one embodiment, HDA comprises or consists of SEQ ID NO: 1, wherein X₁ is H or Q, X₂ is K or Q and X₃ is N or K; or a variant thereof with an amino acid sequence having at least 80%, 85%, 90%, 95%, 96%, 97%, 98% identity to SEQ ID NO: 1; and/or HDB comprises or consists of SEQ ID NO: 2, wherein X₄ is Y or F; X₅ is L or V; X₆ is A or G; X₇ is K or Q; X₈ is N or K; or a variant thereof with an amino acid sequence with at least 80%, 85%, 90%, 95%, 96%, 97%, 98% identity to SEQ ID NO: 2, wherein SEQ ID NO: 1 or its variant can heterodimerize with SEQ ID NO: 2 or its variant. In instances where HDA comprises or consists of a variant of SEQ ID NO: 1, said variant of SEQ ID NO: 1 does not comprise a mutation at position 24, in some embodiments said variant does not comprise a mutation at position 24 and no more than 1 or 2 mutations at positions 3, 5, 22, 23, 25, 26, 36, 38 and 40, in some embodiments said variant does not comprise a mutation at positions 3, 5, 22-26, 36, 38 and 40. In instances where HDB comprises or consists of a variant of SEQ ID NO: 2, said variant of SEQ ID NO: 2 does not comprise a mutation at position 25, in some embodiments said variant does not comprise a mutation at position 25 and no more than 1 or 2 mutations at positions 4, 6-10, 17, 23, 24, 26 and 27, in some embodiments said variant does not comprise a mutation at positions 4, 6-10, 17 and 23-27.

In one embodiment, HDA comprises or consists of SEQ ID NO: 3 or a variant thereof with an amino acid sequence having at least 80%, 85%, 90%, 95%, 96%, 97%, 98% identity to SEQ ID NO: 3; and HDB comprises or consists of SEQ ID NO: 4 or a variant thereof with an amino acid sequence with at least 80%, 85%, 90%, 95%, 96%, 97%, 98% identity to SEQ ID NO: 4, wherein SEQ ID NO: 3 or its variant can heterodimerize with SEQ ID NO: 4 or its variant.

In one embodiment, HDA comprises or consists of SEQ ID NO: 5 or a variant thereof with an amino acid sequence having at least 80%, 85%, 90%, 95%, 96%, 97%, 98% identity to SEQ ID NO: 5; and HDB comprises or consists of SEQ ID NO: 6 or a variant thereof with an amino acid sequence with at least 80%, 85%, 90%, 95%, 96%, 97%, 98% identity to SEQ ID NO: 6, wherein SEQ ID NO: 5 or its variant can heterodimerize with SEQ ID NO: 6 or its variant.

In one embodiment, HDA comprises or consists of SEQ ID NO: 7 or a variant thereof with an amino acid sequence having at least 80%, 85%, 90%, 95%, 96%, 97%, 98% identity to SEQ ID NO: 7; and HDB comprises or consists of SEQ ID NO: 8 or a variant thereof with an amino acid sequence with at least 80%, 85%, 90%, 95%, 96%, 97%, 98% identity to SEQ ID NO: 8, wherein SEQ ID NO: 7 or its variant can heterodimerize with SEQ ID NO: 8 or its variant.

In one embodiment, HDA comprises or consists of SEQ ID NO: 9 or a variant thereof with an amino acid sequence having at least 80%, 85%, 90%, 95%, 96%, 97%, 98% identity to SEQ ID NO: 9; and HDB comprises or consists of SEQ ID NO: 10 or a variant thereof with an amino acid sequence with at least 80%, 85%, 90%, 95%, 96%, 97%, 98% identity to SEQ ID NO: 10, wherein SEQ ID NO: 9 or its variant can heterodimerize with SEQ ID NO: 10 or its variant.

In instances where HDA comprises or consists of a variant of SEQ ID NO: 3, 5, 7 or 9 said variant of SEQ ID NO: 3, 5, 7 or 9 does not comprise a mutation at position 24, in some embodiments said variant does not comprise a mutation at position 24 and no more than 1 or 2 mutations at positions 3, 5, 22, 23, 25, 26, 36, 38 and 40, in some embodiments said variant does not comprise a mutation at positions 3, 5, 22-26, 36, 38 and 40. In instances where HDB comprises or consists of a variant of SEQ ID NO: 4, 6, 8 or 10, said variant of SEQ ID NO: 4, 6, 8 or 10 does not comprise a mutation at position 25, in some embodiments said variant does not comprise a mutation at position 25 and no more than 1 or 2 mutations at positions 4, 6-10, 17, 23, 24, 26 and 27, in some embodiments said variant does not comprise a mutation at positions 4, 6-10, 17 and 23-27.

The determination of percent identity between two sequences is accomplished using the mathematical algorithm of Karlin and Altschul, Proc. Natl. Acad. Sci. USA 90, 5873-5877, 1993. Such an algorithm is incorporated into the BLASTN and BLASTP programs of Altschul et al. (1990) J. Mol. Biol. 215, 403-410. To obtain gapped alignments for comparative purposes, Gapped BLAST is utilized as described in Altschul et al. (1997) Nucleic Acids Res. 25, 3389-3402. When utilizing BLAST and Gapped BLAST programs, the default parameters of the respective programs are used. Alternatively, a variant can also be defined as having up to 20, 15, 10, 5, 4, 3, 2, or 1 amino acid substitutions, in some embodiments said amino acid substitutions are conservative amino acid substitutions. Conservative substitutions are well known in the art (see for example Creighton (1984) Proteins. W.H. Freeman and Company). An overview of physical and chemical properties of amino acids is given in Table 1 below. In one embodiment, conservative substitutions are substitutions made with amino acids having at least one property according to Table 1 in common (i.e. of column 1 and/or 2).

TABLE 1
Properties of naturally occurring amino acids.

| Charge properties/ | | |
|---|---|---|
| hydrophobicity | Side group | Amino Acid |
| nonpolar hydrophobic | aliphatic | Ala, Ile, Leu, Val |
| | aliphatic, S-containing | Met |
| | aromatic | Phe, Trp |
| | imino | Pro |
| polar uncharged | aliphatic | Gly |
| | amide | Asn, Gln |
| | aromatic | Tyr |
| | hydroxyl | Ser, Thr |
| | sulfhydryl | Cys |
| positively charged | basic | Arg, His, Lys |
| negatively charged | acidic | Asp, Gly |

In one embodiment, SEQ ID NO 2 further comprises one or more Lys residues at its C-terminus as an aa addition. In one embodiment, SEQ ID NO 2 further comprises a Ser residue and a Lys residue (i.e. SK) at its C-terminus as an aa addition. In one embodiment, SEQ ID NO 2 further comprises the residues SKTK or SKAK at its C-terminus.

In one embodiment, SEQ ID NO 4, 6, 8 or 10 further comprise one or more Lys residues at their respective C-terminus as an aa addition. In one embodiment, SEQ ID NO 4, 6, 8 or 10 further comprise a Ser residue and a Lys residue (i.e. SK) at their respective C-terminus as an aa addition. In one embodiment, SEQ ID NO 4, 6, 8 or 10 further comprise the residues SKTK or SKAK at their respective C-terminus.

In one embodiment, the complex comprises one or more antigen binding sites within the PCA and/or the PCB. In one embodiment, each of the one or more antigen binding sites is formed by a pair of two variable domains, wherein one is comprised in the PCA and the other is comprised in the PCB. In the context of the present specification, an antigen binding site may also be referred to as paratope. In certain embodiments of the first aspect of the invention, the protein complex is an antibody or antibody-like molecule.

In one embodiment, the antigen binding site comprises or consists of one or two variable domains. In one embodiment, the antigen binding site comprises or consists of two variable domains.

The antigen binding site may comprise or consist of a variable domain of a heavy chain of a Camelidae immunoglobulin. In one embodiment, the antigen binding site comprises or consists of a variable domain of a light chain and a variable domain of a heavy chain. In one embodiment, the antigen binding site comprises or consists of a variable domain of an alpha chain and a variable domain of a beta chain. The antigen binding site may also comprise or consist of one or two variable domains each formed by a protein scaffold, for example an adnectin, an affilin, an affimer, an affitin, an alphabody, an anticalin, an armadillo repeat protein-based scaffold, an atrimer, an avimer, a fynomer, a Kunitz domain, a knottin, an affibody, a β-hairpin mimetic, a monobody, a nanofitin or an ankyrin repeat protein (DARPins).

In one embodiment, the PCA and/or the PCB comprises one or more further homo and/or heterodimerization domain(s) C (HDC). An HDC mediates homo or heterodimerization with a second HDC comprised in another polypeptide chain, i.e. two HDC will bind to each other.

In one embodiment, PCA and PCB each comprise further heterodimerization domains. An example for a configuration in which PCA and PCB each comprise one further heterodimerization domain HDC is the DVD-Fab split CH2 format described herein (variant 2, FIG. 2B) or the CODV-Fab split CH2 format described herein (variant 3, FIG. 2C). In examples of these formats, one HDC is a CH1 domain and the other is a CL domain.

In one embodiment, one of PCA or PCB comprises a further homodimerization domain. An example for a configuration in which only one of PCA or PCB but not the other comprises a homodimerization domain HDC are the symmetric, tetravalent bispecific antibody formats described herein (p. 22 paragraph 2). In these configurations, the polypeptide chain comprising the homodimerization domain HDC, e.g. PCA, binds to a second PCA identical to the first PCA. Second and first PCA each bind to a PCB via the interaction of the heterodimerization domains HDA and HDB. Thus, a tetramer is formed. In examples of these formats, HDC is a CH2-CH3 domain.

An example for a configuration in which only one of PCA or PCB but not the other comprises a homodimerization domain and in addition both of PCA and PCB comprise a further heterodimerization domain (in addition to HDA or HDB, respectively) are the DVD and CODV configuration tetravalent bispecific antibodies described herein (variants 9 and 10, FIG. 5B,C).

In one embodiment, one of PCA or PCB comprises a further heterodimerization domain. An example for a configuration in which only one of PCA or PCB but not the other comprises a further heterodimerization domain HDC are the asymmetric, bivalent bispecific antibody formats described herein (variant 4, FIG. 2D). In examples of these formats, HDC is a knob-into-hole CH2-CH3 domain.

In one embodiment, the homodimerization domain is selected from the group consisting of a CH3 domain, a CH2-CH3 domain, or a domain where homodimerization is mediated by an Ig-like fold, a rossmann- or rossmann-like alpha-beta-alpha sandwich fold, an alpha-sandwich fold, a continuous-beta-sheet fold, a beta-sandwich fold, a mixed beta-sheet fold, a 2-helix orientation, an antiparallel alpha-helix-orientation, a parallel alpha-helix orientation, a 4-helix bundle motif, a leucine zipper and a coiled-coil domain.

In one embodiment, the heterodimerization domain is selected from the group consisting of a knob-into-hole CH3 domain, a knob-into-hole CH2-CH3 domain, a Fc-domain with introduced mutations to force heterodimerization (e.g. charged mutations), a domain of a pair of interchanged domains (such as Fc-one/kappa heterodimerization domain, CL and CH domains), an Ig-like fold with introduced mutations to force heterodimerization, or a domain mediating heterodimerization containing a rossmann- or rossmann-like alpha-beta-alpha sandwich fold, an alpha-sandwich fold, a continuous-beta-sheet fold, a beta-sandwich fold, a mixed beta-sheet fold, a 2-helix orientation, an antiparallel alpha-helix-orientation, a parallel alpha-helix orientation, a 4-helix bundle motif, a leucine zipper and a coiled-coil domain.

In the context of the present specification, a “knob-into-hole CH3 domain” refers to one of a pair of CH3 domains comprising “knob-into-hole” mutations. Such knob-into-hole mutations are amino acid substitutions in order to create a “knob” on one CH3 domain and a “hole” on the other CH3 domain. The knob is represented by a tyrosine (Y), whereas the hole is represented by a threonine (T). In particular, knob-into-hole mutations are T366Y in one CH3 domain and Y407T in the other, wherein the two CH3 domains are IgG1 constant domains, and optionally wherein the Fc region comprising the T366Y mutation (“knob” chain) further comprises the mutations S354C and T166W and the Fc region comprising the Y407T mutation (“hole” chain) further comprises the mutations Y349C, T366S, L368A and Y407V.

In the context of the present specification, the term “RF mutations” refers to mutations H435R and Y436F (RF mutations) in one of a pair of CH3 domains. The RF mutations may be in a CH3 domain comprising a T366Y mutation (“knob” chain) or in a CH3 domain comprising a Y407T mutation (“hole” chain).

In the context of the present specification, a “knob-into-hole CH2-CH3 domain” is a CH2-CH3 domain wherein the CH3 domain comprises “knob-into-hole” mutations.

In one embodiment, the antigen binding site(s) are located N- and/or C-terminally of the HDA or HDB.

In one embodiment, the antigen binding site(s) are located N- and/or C-terminally of the HDC.

In one embodiment, PCA and PCB comprise from N- to C-terminus the following elements:
(i) PCA: V2-L1-HDA, and PCB: V2-L2-HDB;
(ii) PCA: V1-L3-HDA-L4-V2, and PCB: V1-L1-HDB-L2-V2;
(iii) PCA: V1-L1-V2-L2-HDA, and PCB: V2-L3-V1-L4-HDB;
(iv) PCA: V1-L3-V2-L5-CL-L4-HDA, and PCB: V1-L1-V2-L6-CH1-L2-HDB; wherein L5, CL, L6 and CH1 may be present or absent; or
(v) PCA: V1-L4-V2-L5-CL-L6-HDA, and PCB: V2-L1-V1-L2-CH1-L3-HDB; wherein L5, CL, L2 and CH1 may be present or absent.

In all embodiments (i)-(v) of PCA and PCB mentioned in the above paragraph, each pair of V1, V2, V3, and V4 (i.e. V1/V1, V2/V2, V3/V3, and V4/V4) comprises or consists of a variable domain of a heavy chain and a variable domain of a light chain or a variable domain of an alpha chain and a variable domain of a beta chain and forms an antigen binding site. L1 to L6 are peptide linker. CH1 is a heavy chain constant domain 1. CL is a light chain constant domain. The PCA and/or the PCB may further comprise an HDC.

The term “peptide linker” generally and unless specified otherwise refers to a moiety that couples two domains by forming a peptide bond with each of the domains. Herein, it is referred to a linker having a length of 0-X amino acids (“aa”). If two domains are coupled by a linker having a length of 0 aa, this means that the two domains are linked directly via a peptide bond between the two domains. For this also the term “fused” instead of linked may be used. A peptide linker is in particular a flexible peptide linker, i.e. it provides flexibility among the domains that are linked together. Such flexibility is generally increased if the amino acids are small and do not have bulky side chains that impede rotation or bending of the amino acid chain. Thus, in some embodiments, the peptide linker of the present invention has an increased content of small amino acids, for example of glycines, alanines, serines, threonines, leucines and isoleucines. In some embodiments, at least 20%, 30%, 40%, 50%, 60% 70%, 80%, 90% or more of the amino acids of the peptide linker are such small amino acids. In one embodiment, the amino acids of the linker are selected from glycines and serines, i.e. said linker is a poly-glycine or a poly-glycine/serine linker, wherein “poly” means a proportion of at least 50%, 60%, 70%, 80%, 90% or even 100% glycine and/or serine residues in the linker. In the context of the present specification, the term poly-glycine/serine linker may also refer to a linker consisting of only one amino acid selected from G or S.

In some embodiments, the peptide linkers L1 to L6 comprise or consist of an amino acid sequence of the general formula (I): [G_{w}SₓG_{y}]_{z} (SEQ ID NO: 34), wherein w is an integer between 0 and 20, in some embodiments between 2 and 5, x is an integer between 0 and 10, in some embodiments between 0 and 3, y is an integer between 0 and 20, in some embodiments between 0 and 5 and z is an integer between 0 and 10, in some embodiments between 0 and 4. A peptide linker length of 20 amino acids (aa) or less is particularly useful.

In embodiment (i), PCA and PCB comprise from N- to C-terminus the following elements: PCA: V2-L1-HDA, and PCB: V2-L2-HDB. In some embodiments, one of PCA or PCB further comprises a heterodimerization domain HDC. Examples for this configuration are the PCA and PCB comprised in the asymmetric, bivalent bispecific antibody formats described herein (variants 11 and 12, FIG. 5D, E). In some embodiments, L1 and L2 have a length of 20 aa or less, in some embodiments 15 aa or less, in some embodiments 10 aa or less, in some embodiments 5 aa or less. In some embodiments, L1 and L2 are poly-glycine or poly-glycine/serine linker. In a non-limiting example, PCB further comprises a heterodimerization domain HDC, L1 is G₂ and L2 is (G₅S)₂ (SEQ ID NO: 35).

In embodiment (ii), PCA and PCB comprise from N- to C-terminus the following elements: PCA: V1-L3-HDA-L4-V2, and PCB: V1-L1-HDB-L2-V2. An example for this configuration is the “splite” (“split bite-like”) diabody format described herein (variants 4-7, FIG. 2D). In the “splite” diabody format, HDA and HDB have been inserted between the variable domains V1 and V2 of PCA and PCB, respectively (FIG. 2D). In some embodiments, PCA and PCB comprise from N- to C-terminus the following elements: PCA: VL1-L3-HDA-L4-VL2, and PCB: VH1-L1-HDB-L2-VH2. In some embodiments of the “splite” diabody format, L1 to L4 have a length of 20 aa or less, in some embodiments 15 aa or less. In some embodiments, L1 to L4 are poly-glycine or poly-glycine/serine linker. In some embodiments, two or three of L1 to L4 are 0 aa.

In some embodiments of the “splite” diabody format,
L3 is has a length of 10 to 20 aa, in some embodiments 13 to 17 aa, in some embodiments about 15 aa, L4 has a length of 0-10 aa, in some embodiments 0-5 aa, in some embodiments 0 aa, L1 has a length of 10 to 20 aa, in some embodiments 13 to 17 aa, in some embodiments about 15 aa and L2 has a length of 0-10 aa, in some embodiments 0-5 aa, in some embodiments 0 aa;
L3 has a length of 0-10 aa, in some embodiments 0-5 aa, in some embodiments about 3 aa and L4, L1 and L2 have a length of 0-10 aa, in some embodiments 0-5 aa, in some embodiments 0 aa;
L3 has a length of 0-10 aa, in some embodiments 0-5 aa, in some embodiments about 3 aa, L1 has a length of 10 to 20 aa, in some embodiments 13 to 17 aa, in some embodiments about 15 aa and L4 and L2 have a length of 0-10 aa, in some embodiments 0-5 aa, in some embodiments 0 aa; or
L3 has a length of 10 to 20 aa, in some embodiments 13 to 17 aa, in some embodiments about 15 aa, and L4, L1 and L2 have a length of 0-10 aa, in some embodiments 0-5 aa, in some embodiments 0 aa.

By way of non-limiting example, possible combinations of L1 to L4 are: L3 is (G₄S)₃ (SEQ ID NO: 36), L4 is 0 aa, L1 is (G₄S)₃ (SEQ ID NO: 36) and L2 is 0 aa; L3 is G₃, L4 is 0 aa, L1 is 0 aa and L2 is 0 aa; L3 is G₃, L4 is 0 aa, L1 is (G₄S)₃ (SEQ ID NO: 36) and L2 is 0 aa or L3 is (G₄S)₃ (SEQ ID NO: 36), L4 is 0 aa, L1 is 0 aa and L2 is 0 aa.

The formats according to embodiment (ii) may also comprise additional pairs of variable domains, e.g. two pairs of variable domains on each side (i.e. N-terminally and C-terminally) of the reconstituted split Ig domain, resulting in a tetravalent construct.

In embodiment (iii), PCA and PCB comprise from N- to C-terminus the following elements: PCA: V1-L1-V2-L2-HDA, and PCB: V2-L3-V1-L4-HDB. An example for this configuration is the diabody split CH2 format as described herein (variant 1, FIG. 2A). In the diabody split CH2 format, the variable domains of PCA and PCB are oriented crosswise, i.e. V1 is located N-terminally to V2 in PCA, and C-terminally to V2 in PCB. Thus, in the diabody split CH2 format the linkers L1 to L4 have to provide sufficient mobility for the variable domains to fold into a cross-over configuration. HDA and HDB have been added C-terminally to the variable domains of PCA and PCB, respectively (FIG. 2A). In some embodiments, PCA and PCB comprise from N- to C-terminus the following elements: PCA: VL1-L1-VH2-L2-HDA, and PCB: VL2-L3-VH1-L4-HDB. In some embodiments of the diabody split CH2 format, L1 to L4 have a length of 20 aa or less. In some embodiments, L2, L3 and L4 have a length of 5 to 20 aa and L2 has a length of 0 to 5 aa. In some embodiments, L1 to L4 are poly-glycine or poly-glycine/serine linker or 0 aa. In some embodiments, L1 and L3 have a length of 5 to 15 aa, in some embodiments 5 to 10 aa, in some embodiments about 8 aa, L2 has a length of 0-10 aa, in some embodiments 0-5 aa, in some embodiments 0 aa and L4 has a length of 5 to 20 aa, in some embodiments 8 to 15 aa, in some embodiments about 10 aa.

In a non-limiting example of the diabody split CH2 format, L1 and L3 are G₃SG₄ (SEQ ID NO: 37), L2 is 0 aa and L4 is (G₄S)₂ (SEQ ID NO: 38).

In embodiment (iv), PCA and PCB comprise from N- to C-terminus the following elements: PCA: V1-L3-V2-L5-CL-L4-HDA, and PCB: V1-L1-V2-L6-CH1-L2-HDB; wherein L5, CL, L6 and CH1 may be present or absent. An example for this configuration is the DVD-Fab split CH2 format described herein (variant 2, FIG. 2B). In the DVD-Fab split CH2 format, PCA and PCB each comprise a constant heterodimerization domain (CH1 or CL, respectively) C-terminally of the variable domains and N-terminally of HDA or HDB, respectively (FIG. 2B). In some embodiments, PCA and PCB comprise from N- to C-terminus the following elements: PCA: VL1-L3-VL2-L5-CL-L4-HDA, and PCB: VH1-L1-VH2-L6-CH1-L2-HDB. In some embodiments of the DVD-Fab split CH2 format, L1 to L6 have a length of 20 aa or less. In some embodiments, L1 to L6, in some embodiments L1 to L3 are poly-glycine or poly-glycine/serine linker or 0 aa. In some embodiments, L5 and L6 are 0 aa. In some embodiments, L1 has a length of 5 to 15 aa, in some embodiments about 10 aa, L2 has a length of 0-10 aa, in some embodiments 0-5 aa, in some embodiments about 3 aa, L3 has a length of 5 to 15 aa, in some embodiments about 10 aa, L4 has a length of 0-10 aa, in some embodiments 0-5 aa, in some embodiments 0 aa, and L5 and L6 have a length of 0-10 aa, in some embodiments 0-5 aa, in some embodiments 0 aa.

In a non-limiting example of the DVD-Fab split CH2 format, L1 is (G₄S)₂ (SEQ ID NO: 38), L2 is G₃, L3 is (G₄S)₂ (SEQ ID NO: 38), and L4 to L6 are 0 aa.

In embodiment (v), PCA and PCB comprise from N- to C-terminus the following elements: PCA: V1-L4-V2-L5-CL-L6-HDA, and PCB: V2-L1-V1-L2-CH1-L3-HDB; L5, CL, L2 and CH1 may be present or absent. An example for this configuration is the CODV-Fab split CH2 format as described herein (variant 3, FIG. 2C). In the CODV-Fab split CH2 format, the variable domains of PCA and PCB are oriented crosswise, i.e. V1 is located N-terminally to V2 in PCA, and C-terminally to V2 in PCB. Thus, in the CODV-Fab split CH2 format the linkers L1 to L6 have to provide sufficient mobility for the variable domains to fold into a cross-over configuration. In addition, PCA and PCB each comprise a constant heterodimerization domain (CH1 or CL, respectively) C-terminally of the variable domains and N-terminally of HDA or HDB, respectively (FIG. 2C). In some embodiments, PCA and PCB comprise from N- to C-terminus the following elements: PCA: VL1-L4-VL2-L5-CL-L6-HDA, and PCB: VH2-L1-VH1-L2-CH1-L3-HDB. In some embodiments of the CODV-Fab split CH2 format, L1 to L6 have a length of 20 aa or less. In some embodiments, L4 and L5 have a length of 5 to 15 aa and L1 and L2 have a length of 10 aa or less. In some embodiments, L1 to L6 are poly-glycine, poly-serine, poly-glycine/serine linker. In some embodiments, L3 is a poly-glycine, poly-serine, poly-glycine/serine linker. In some embodiments, L4 has a length of 5 to 15 aa, in some embodiments 5 to 10 aa, in some embodiments about 8 aa, L5 has a length of 5 to 15 aa, in some embodiments 5 to 10 aa, in some embodiments about 6 aa, L1 has a length of 0-10 aa, in some embodiments 0-5 aa, in some embodiments about 1 aa, L2 has a length of 0-10 aa, in some embodiments 0-5 aa, in some embodiments 0 aa, L6 has a length of 0-10 aa, in some embodiments 0-5 aa, in some embodiments 0 aa and L3 has a length of 0-10 aa, in some embodiments 0-5 aa, in some embodiments about 3 aa.

In a non-limiting example of the CODV-Fab split CH2 format, L4 is GQPKAAPS (SEQ ID NO: 39), L5 is TKGPSV (SEQ ID NO: 40), L1 is S, L2 is 0 aa, L6 is 0 aa and L3 is G₃.

The formats according to embodiments (iii) to (v) may also comprise additional pairs of variable domains, e.g. a total of three pairs of variable domains, resulting in a trivalent construct.

In one embodiment, the PCA and/or the PCB comprises a first HDC and the complex comprises one or more additional polypeptides comprising one or more antigen binding sites and a second HDC that is covalently or non-covalently bound to the first HDC. In some embodiments, the additional polypeptides have a structure according to PCA and/or PCB or an antibody-like structure.

Examples of configurations in which the additional polypeptides have a structure according to PCA and PCB are the symmetric, tetravalent bispecific antibody formats described herein (variants 8-10, FIG. 5A-C). These symmetric formats comprise the PCA and PCB (first PCA and first PCB) according to the embodiments described above, in embodiments (iii) to (v), a second PCA identical to the first PCA and a second PCB identical to the first PCB. Homodimerization of the first and second PCA-PCB pair is mediated by covalent or non-covalent, for example non-covalent, binding of a homodimerization domain HDC comprised in the first pair of PCA and PCB to a homodimerization domain HDC comprised in the second pair of PCA and PCB.

The diabody split CH2 tetravalent bispecific antibody format (variant 8) comprises two identical PCA and two identical PCB as described above for the diabody format (variant 1), wherein the two PCA or the two PCB, in some embodiments the two PCB, further comprise a homodimerization domain HDC mediating homodimerization of the two PCA or PCB, in some embodiments the two PCB.

The DVD split CH2 tetravalent bispecific antibody format (variant 9) comprises two identical PCA and two identical PCB as described above for the DVD-Fab split CH2 format (variant 2), wherein the two PCA or the two PCB, in some embodiments the two PCB, further comprise a homodimerization domain HDC mediating homodimerization of the two PCA or PCB, in some embodiments the two PCB.

The CODV split CH2 tetravalent bispecific antibody format (variant 10) comprises two identical PCA and two identical PCB as described above for the CODV-Fab split CH2 format (variant 3), wherein the two PCA or the two PCB, in some embodiments the two PCB, further comprise a homodimerization domain HDC mediating homodimerization of the two PCA or PCB, in some embodiments the two PCB.

Another example of a configuration in which the additional polypeptides have a structure according to PCA and PCB are asymmetric antibody formats, in which the second PCA and second PCB are different from the first PCA and first PCB.

In the context of the present specification, an antibody-like structure can be an antibody derivative or an antibody mimetic. Examples of configurations in which the additional polypeptides have an antibody-like structure are the asymmetric, split CH2 bivalent bispecific antibody formats described herein (variants 11-12, FIG. 5D-E). These formats comprise the PCA and PCB according to embodiment (i) as described above and an antibody heavy chain and an antibody light chain covalently or non-covalently bound to each other. Binding between PCA/PCB and the antibody heavy chain is mediated by covalent or non-covalent, in one embodiment non-covalent, binding of the first HDC comprised in PCA or PCB to the second HDC comprised in the antibody heavy chain. In this configuration, the first and the second HDC are in some embodiments hetero dimerization domains that overcome the heavy chain pairing problem. In one embodiment, the first HDC and the second HDC comprise knob-into-hole CH3 domains. One embodiment of this format can also be described as Fab/Fv split CH2 IgG, wherein one arm of the IgG is a monovalent Fab and the other arm is a monovalent Fv split CH2 comprising a PCA consisting of V2-L1-HDA and a PCB consisting of V2-L2-HDB.

In one embodiment, the protein complex is monovalent and monospecific, bivalent and mono- or bispecific, trivalent and mono-, bi- or trispecific, tetravalent and mono-, bi-, tri- or tetraspecific, pentavalent and mono-, bi-, tri-, tetra- or pentaspecific, or hexavalent and mono-, bi-, tri-, tetra-, penta- or hexaspecific.

In one embodiment, the protein complex is tetravalent and bispecific.

In one embodiment, the protein complex is bivalent and bispecific.

The reassembly of an Ig domain was used by the inventors to address the light chain pairing problem occurring during the production of bivalent bispecific antibodies. The inventors found that in one arm of a bivalent bispecific antibody, a HDA and a HDB as described above can be used to replace the CH1 domain of the heavy chain and the CL domain of the light chain, respectively. Consequently, the light and heavy chains comprising HDA and HDB will heterodimerize to form a first antibody arm. For the second arm of said bivalent bispecific antibody, the non-modified light chain will heterodimerize with the non-modified heavy chain via the interaction of the CH1 domain and the CL domain. Neither CH1 nor CL will dimerize with HDA or HDB. Thus, both arms are assembled without undesired light chain pairings. Finally, heterodimerization of the first arm and the second arm can be controlled by one of the techniques known in the art to overcome the heavy chain pairing problem.

A multispecific antibody or derivative is capable of binding multiple different antigens and a bispecific antibody or derivative is capable of binding two different antigens.

In some embodiments of all antibodies or derivatives described herein, the antibody or derivative is capable of binding to IL-4 and/or IL-13. In some embodiments, the antibody or derivative is bispecific and capable of binding to IL-4 and IL-13.

Examples for a symmetric multispecific antibody of the invention are a diabody-type tetravalent bispecific antibody (FIG. 5A), a DVD configuration tetravalent bispecific antibody (FIG. 5B) or a CODV configuration tetravalent bispecific antibody (FIG. 5C).

An example for an asymmetric multispecific antibody of the invention is a bivalent bispecific antibody (FIGS. 5D and E), or the diabody, DVD-Fab split CH2, CODV-Fab split CH2 or “splite” diabody (FIG. 2A to D).

In certain embodiments, the antibody or derivative thereof may have reduced or no Fc effector functions. An Fc effector function is the interaction with complement protein C1q and/or the binding to Fc receptors. A reduced or a lack of effector functions can be achieved for example by a double mutation L234A and L235A (so-called “LALA mutation”) in the CH2A and/or the CH2B domain. Corresponding mutations can also be introduced in HDA and HDB if the respective N-β and C-β comprise an amino acid sequence of a CH2 domain.

All terms used with respect to the following second, third, fourth and fifth aspects of the invention have the meanings as defined with respect to the first aspect of the invention, unless specifically defined otherwise. Further, all embodiments specified for the first aspect that are applicable to the second, third, fourth and fifth aspects are also envisaged for those aspects.

In a second aspect, the present invention relates to one or more polynucleotides encoding the at least two polypeptides of the protein complex according to the first aspect of the invention. The one or more polynucleotides according to the second aspect may also encode the one or more additional polypeptides comprised in the protein complex according to the first aspect. In one embodiment, the one or more polynucleotides encode an antibody or antibody-like structure. This refers to all embodiments described above, for instance to any of the antibody or antibody derivative configurations described herein. In one embodiment, the one or more polynucleotides are isolated.

In a third aspect, the present invention relates to one or more expression vectors comprising the one or more polynucleotides according to the second aspect of the invention. The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been fused. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes they comprise. Such vectors are referred to herein as “expression vectors”.

In a fourth aspect, the present invention relates to a cell comprising the one or more polynucleotides according to the second aspect of the invention or the one more expression vectors according to the third aspect of the invention. A wide variety of cell expression systems can be used to express said polynucleotides including the use of prokaryotic and eukaryotic cells, such as bacterial cells (e.g. E. coli), yeast cells, insect cells or mammalian cells (e.g. mouse cells, rat cells, human cells etc.). For this purpose, a cell is transformed or transfected with said polynucleotide(s) or expression vector(s) such that the polynucleotide(s) of the invention are expressed in the cell and, in one embodiment, secreted into the medium in which the cells are cultured, from where the expression product can be recovered.

In a fifth aspect, the present invention relates to a pharmaceutical composition comprising a pharmaceutically acceptable carrier and the protein complex according to the first aspect of the invention, the one or more polynucleotides according to the second aspect of the invention or the one or more expression vectors according to the third aspect of the invention. In one embodiment, the protein complex is an antibody or antibody-like molecule that specifically binds to a pathogen, a diseased cell, a cell receptor or a cell signaling molecule. The pharmaceutical compositions of the invention can be selected for parenteral delivery. Alternatively, the compositions can be selected for inhalation or for delivery through the digestive tract, such as orally. The preparation of such pharmaceutically acceptable compositions is within the skill of the art.

The term “pharmaceutically acceptable carrier” or “physiologically acceptable carrier” as used herein refers to one or more formulation materials suitable for accomplishing or enhancing the delivery of an antibody or antibody-like molecule. The primary carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the invention, antibody or antibody-like molecule compositions can be prepared for storage by mixing the selected composition having the desired degree of purity with optional formulation agents in the form of a lyophilized cake or an aqueous solution. Further, the antibody or antibody-like molecule can be formulated as a lyophilizate using appropriate excipients such as sucrose.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediaminetetraacetic acid (EDTA)), complexing agents (such as caffeine, polyvinylpyrrolidone, beta-cyclodextrin, or hydroxypropylbeta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, for example sodium or potassium chloride—or mannitol sorbitol), delivery vehicles, diluents, excipients and/or pharmaceutical adjuvants (see, e.g., REMINGTON's PHARMACEUTICAL SCIENCES (18th Ed., A.R. Gennaro, ed., Mack Publishing Company 1990), and subsequent editions of the same).

The term “specifically binds” as used herein refers to a binding reaction which is determinative of the presence of the target molecule in vitro or in vivo, for instance in an organism such as the human body. As such, the specified ligand binds to its particular target molecule and does not bind in a substantial amount to other molecules present. Generally, an antibody or derivative thereof that “specifically binds” a target molecule has an equilibrium affinity constant greater than about 105 (e.g., 106, 107, 108, 109, 1010, 1011, and 1012 or more) mole/liter for that target molecule.

The term “pathogen” refers to any organism which may cause disease in a subject. It includes but is not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host, but are in an immunocompromised host.

The diseased cell may be a tumor cell, a chronically infected cell, a senescent cell, a cell showing an inflammatory phenotype, a cell accumulating amyloid proteins or a cell accumulating misfolded proteins.

In case of a tumor cell, the underlying disease is a tumor, for example a tumor associated with IL4/IL13 signaling, such as Hodgkin Lymphoma.

In case of a senescent cell, the underlying disease is a senescence associated disease, such as Idiopathic Pulmonary Fibrosis (IPF) and Chronic Obstructive Pulmonary Disease.

In case of a cell showing an inflammatory phenotype, the underlying disease is an (auto)inflammatory disease, such as an Allergy, Allergic Rhinitis, Asthma, Atopic Dermatitis, Crohn's Disease (CD), Inflammatory Bowel Disease, Systemic Lupus Erithematosus, Systemic Sclerosis and Ulcerative Colitis (UC).

The term “cell receptor” is not limited to any particular receptor. For example, it may be a G-protein coupled receptor, an ion channel or a cross-membrane transporter. Specific examples are CD3, CD4, CD8, CD28, CD16, and NKp46.

The cell signaling molecule may be a cytokine, such as a chemokine, an interferon, an interleukin, a lymphokine or a tumor necrosis factor, or a hormone or growth factor, or a molecule of an intracellular signaling cascade.

In one embodiment of the fifth aspect, the antibody or antibody-like molecule is multispecific, in one embodiment bispecific, and further binds to an effector molecule, e.g. a cytotoxic substance or a receptor ligand.

### Exemplary Formats

Some exemplary antibodies or derivatives thereof according to the invention are represented by amino acid sequences as follows:
Example of variant 1: A diabody split CH2 (FIG. 2A, FIG. 7, Table 7) having
   a PCA according to SEQ ID NO: 11, wherein residues 1-111 are light chain variable domain V1 (VL1) and residues 120-242 are heavy chain variable domain V2 (VH2),
   a PCB according to SEQ ID NO: 12, wherein residues 1-107 are light chain variable domain V2 (VL2) and residues 116-233 are heavy chain variable domain V1 (VH1).
Example of variant 2: A DVD-Fab split CH2 (FIG. 2B, FIG. 8, Table 8, 9) having
   a PCA according to SEQ ID NO: 13, wherein residues 1-111 are light chain variable domain V1 (VL1) and residues 122-228 are light chain variable domain V2 (VL2),
   a PCB according to SEQ ID NO: 14, wherein residues 1-118 are heavy chain variable domain V1 (VH1) and residues 120-251 are heavy chain variable domain V2 (VH2).
Example of variant 3: A CODV-Fab split CH2 (FIG. 2C, FIG. 9, Table 10) having
   a PCA according to SEQ ID NO: 15, wherein residues 1-111 are light chain variable domain V1 (VL1) and residues 120-226 are light chain variable domain V2 (VL2),
   a PCB according to SEQ ID NO: 16, wherein residues 1-133 are heavy chain variable domain V2 (VH2) and residues 135-242 are heavy chain variable domain V1 (VH1).
   In the CODV format, each of PCA and PCB comprises two variable domains V1 and V2, a constant domain (CL or CH1) and a heterodimerization domain (HDA or HDB). The order of the variable domains from N-term to C-term is V1-V2 in one polypeptide chain and V2-V1 in the other polypeptide chain.
Example of variant 4: A splite diabody (FIG. 2D, FIG. 10, FIG. 19, Table 11) having
   a PCA according to SEQ ID NO: 17, wherein residues 1-111 are light chain variable domain V1 (VL1) and residues 178-284 are light chain variable domain V2 (VL2),
   a PCB according to SEQ ID NO: 18, wherein residues 1-133 are heavy chain variable domain V1 (VH1) and residues 193-315 are heavy chain variable domain V2 (VH2).
Example of variant 5: A splite diabody (FIG. 2D, FIG. 11, Table 12) having
   a PCA according to SEQ ID NO: 19, wherein residues 1-111 are light chain variable domain V1 (VL1) and residues 173-289 are light chain variable domain V2 (VL2),
   a PCB according to SEQ ID NO: 20, wherein residues 1-133 are heavy chain variable domain V1 (VH1) and residues 181-303 are heavy chain variable domain V2 (VH2).
Example of variant 6: A splite diabody (FIG. 2D, FIG. 12, Table 13) having
   a PCA according to SEQ ID NO: 17, wherein residues 1-111 are light chain variable domain V1 (VH1) and residues 178-284 are light chain variable domain V2 (VL2),
   a PCB according to SEQ ID NO: 20, wherein residues 1-133 are heavy chain variable domain V1 (VH1) and residues 181-303 are heavy chain variable domain V2 (VH2).
Example of variant 7: A splite diabody (FIG. 2D, FIG. 13, Table 14) having
   a PCA according to SEQ ID NO: 19, wherein residues 1-111 are light chain variable domain V1 (VL1) and residues 173-289 are light chain variable domain V2 (VL2),
   a PCB according to SEQ ID NO: 18, wherein residues 1-133 are heavy chain variable domain V1 (VH1) and residues 193-315 are heavy chain variable domain V2 (VH2).
Example of variant 8: A diabody split CH2 tetravalent bispecific antibody (FIG. 5A, FIG. 14, Table 15) having
   two PCAs according to SEQ ID NO: 11, wherein residues 1-111 are light chain variable domain V1 (VL1) and residues 120-242 are heavy chain variable domain V2 (VH2),
   two PCBs according to SEQ ID NO: 21, wherein residues 1-107 are light chain variable domain V2 (VL2) and residues 116-233 are heavy chain variable domain V1 (VH1).
Example of variant 9: A DVD split CH2 tetravalent bispecific antibody (FIG. 5B, FIG. 15, Table 16) having
   two PCAs according to SEQ ID NO: 13, wherein residues 1-111 are light chain variable domain V1 (VL1) and residues 122-228 are light chain variable domain V2 (VL2),
   two PCBs according to SEQ ID NO: 22, wherein residues 1-118 are heavy chain variable domain V1 (VH1) and residues 120-251 are heavy chain variable domain V2 (VH2).
Example of variant 10: A CODV split CH2 tetravalent bispecific antibody (FIG. 5C, FIG. 16, Table 17) having
   two PCAs according to SEQ ID NO: 15, wherein residues 1-111 are light chain variable domain V1 (VL1) and residues 120-226 are light chain variable domain V2 (VL2),
   two PCBs according to SEQ ID NO: 23, wherein residues 1-133 are heavy chain variable domain V2 (VH2) and residues 135-242 are heavy chain variable domain V1 (VH1).
Example of variant 11: A split CH2 bivalent bispecific antibody (FIG. 5D, FIG. 17, Table 18) having
   a PCA according to SEQ ID NO: 25, wherein residues 1-107 are light chain variable domain V2 (VL2),
   a PCB according to SEQ ID NO: 26, wherein residues 1-133 are heavy chain variable domain V2 (VH2),
   a light chain according to SEQ ID NO: 27, wherein residues 1-111 are light chain variable domain V1 (VL1),
   a heavy chain according to SEQ ID NO: 24, wherein residues 1-118 are heavy chain variable domain V1 (VH1).
Example of variant 12: A split CH2 bivalent bispecific antibody (FIG. 5E, FIG. 18, Table 19+20) having
   a PCA according to SEQ ID NO: 29, wherein residues 1-133 are heavy chain variable domain V2 (VH2),
   a PCB according to SEQ ID NO: 28, wherein residues 1-107 are light chain variable domain V2 (VL2),
   a light chain according to SEQ ID NO: 27, wherein residues 1-111 are light chain variable domain V1 (VL1),
   a heavy chain according to SEQ ID NO: 24, wherein residues 1-118 are heavy chain variable domain V1 (VH1).
Example of variant 13: A diabody split IgA CH2 (FIG. 21) having
   a PCA according to SEQ ID NO: 41, wherein residues 1-111 are light chain variable domain V1 (VL1) and residues 120-242 are heavy chain variable domain V2 (VH2),
   a PCB according to SEQ ID NO: 42, wherein residues 1-107 are light chain variable domain V2 (VL2) and residues 116-233 are heavy chain variable domain V1 (VH1).
Example of variant 14: A diabody split IgD CH2 (FIG. 22) having
   a PCA according to SEQ ID NO: 43, wherein residues 1-111 are light chain variable domain V1 (VL1) and residues 120-242 are heavy chain variable domain V2 (VH2),
   a PCB according to SEQ ID NO: 44, wherein residues 1-107 are light chain variable domain V2 (VL2) and residues 116-233 are heavy chain variable domain V1 (VH1).
Example of variant 15: A diabody split IgE CH3 having
   a PCA according to SEQ ID NO: 45, wherein residues 1-111 are light chain variable domain V1 (VL1) and residues 120-242 are heavy chain variable domain V2 (VH2),
   a PCB according to SEQ ID NO: 46, wherein residues 1-107 are light chain variable domain V2 (VL2) and residues 116-233 are heavy chain variable domain V1 (VH1).
Example of variant 16: A diabody split IgE CH2 having
   a PCA according to SEQ ID NO: 47, wherein residues 1-111 are light chain variable domain V1 (VL1) and residues 120-242 are heavy chain variable domain V2 (VH2),
   a PCB according to SEQ ID NO: 48, wherein residues 1-107 are light chain variable domain V2 (VL2) and residues 116-233 are heavy chain variable domain V1 (VH1).

In the exemplary formats 1-16 specified above, each pair of light chain and heavy chain variable domains (VL1/VH1, VL2/VH2) may be substituted by a pair of variable domains having a different specificity than the one exemplified.

In the exemplary formats 1-16 specified above, the six C-terminal histidine residues (His-tag) may be absent from SEQ ID NO: 12, SEQ ID NO: 14, SEQ ID NO: 16, SEQ ID NO: 18, SEQ ID NO: 20, SEQ ID NO: 42, SEQ ID NO: 44, SEQ ID NO: 46, and SEQ ID NO: 48.

Due to splitting of the Ig domain in two parts, a new C-terminus is generated in the N-terminal part and a new N-terminus is generated in the C-terminal part. Both the original N- and C-termini and the newly generated N- and C-termini can be used to link different protein domains to the N- and C-terminal part of the Ig domain, such as Fabs, Fvs or Fcs. This enables the generation of multispecific antibodies or derivatives thereof. For example, a tetravalent antibody can be generated by fusing 4 single chain Fv (scFv) each having a different specificity to all 4 termini.

Based on the available structural data it is plausible to argue that using the split Ig domain, in particular CH2 domain, as a building block allows for new spatial arrangements of different fusions partner. In contrast to the consecutive linkage of different or building blocks as used e.g. in multivalent nanobodies, different domains or building blocks are orientated in a crosswise fashion when linked to the split CH2 domains. This might offer new opportunities e.g. to simultaneously blocking different epitopes on one target.

In some embodiments, the heterodimerization domains HDA and HDB consist of the N- and C-terminal part of an Ig domain without any amino acid modifications. In these instances, no new amino acid sequences are introduced, thus minimizing the risk of an immune response against e.g. antibody derivatives comprising HDA and HDB.

In instances where the N-β and C-β are selected from a CH2 domain, for example the same CH2 domain, heterodimerization of HDA and HDB results in the formation of a complete CH2 domain, which can mediate binding to Fc-gamma receptors (FcγR), the neonatal Fc receptor (FcRn), and complement component 1q (C1q). This is particularly advantageous if the protein complex is a multispecific, in particular bispecific, antibody that otherwise does not comprise any constant domain, such as a splite diabody (variants 4-7) or diabody split CH2 (variant 1) as described herein. Binding of the CH2 domain to FcRn is important for medical applications because it leads to an increased plasma half-life. Binding of the CH2 domain to FcγRs and C1q is important for evoking antibody-dependent cell-mediated cytotoxicity (ADCC) or complement-dependent cytotoxicity (CDC), respectively.

An additional advantage of the heterodimerization domains HDA and HDB compared to using two complete Ig domains, as e.g. in the CrossMab format (where the C1 domain from the heavy chain is swapped with the CL domain from a light chain for one Fab-arm), is the reduced size/reduced molecular weight. In oncologic applications, this has the important advantage of an increased tumor penetrance.

### BRIEF DESCRIPTION OF DRAWINGS

FIG. 1: Structural basis for splitting the CH2 domain. (A) Cartoon structure of a full-length human antibody (pdb entry 3 S7G). One CH2 domain is highlighted in black. The glycans are depicted as spheres. Note, that the CH2 domain has no direct protein-protein contacts. (B) Structure of the CH2 domain. The split-site is located within a loop connecting the two β-sheets. The N-terminal half is coloured grey, the C-terminal part in black. A disulfide-bond is formed between the two β-sheets which covalently links the N- with the C-terminus in the split CH2 domain architecture. Cartoons were generated with pymol (https://sourceforge.net/projects/pymol/).

FIG. 2: Schematic structures of Fv-only and Fab-like formats with incorporated split CH2 domain. Upon the CH2 split, a new N- as well as a C-terminus is generated. The N-termini of the different domains are indicated. A his-tag was added in all constructs for purification purposes. (A) Split CH2 domain added to a diabody format (diabody split CH2). (B) DVD-Fab split CH2. (C) CODV-Fab split CH2. (D) Split CH2 domain inserted into a diabody-format (“splite” diabody). In this format different linkers (L1 and L4 respectively) were used at the newly generated N- and C-terminus. The molecules described in this report are numbered consecutively.

FIG. 3: SDS-PAGE of protein variants (1)-(7) analyzed under non-reducing and reducing conditions. All proteins were purified via their His-tag. Since both protein chains are present under reducing conditions, it is obvious that the connection of the two chains occurs via a disulfide-bridge. Protein bands corresponding to the correctly assembled protein are indicated by an arrowhead.

FIG. 4: Stability measurements (1)-(7) and CH2-domain (wt). For all proteins the concentration was adjusted to 0.5 mg/mL. (A) Tryptophan fluorescence measurement allows monitoring the melting of a protein. The measured fluorescence ration F350 nm/F330 nm represents the melting curves of the proteins. The calculated Tₘ (Tab.2) corresponds to the maximum of the first derivative of the F350/F330 curve. (B) Light scattering was used to monitor the aggregation behaviour of the proteins. The calculated T_{agg} (Tab.2) corresponds to the maximum of the first derivative of the measured scatter intensities. Note, that the CH2-domain (wt) does not start to aggregate despite the protein is unfolded.

FIG. 5: Schematic structures different antibody-like formats with incorporated split CH2 domain. Upon the CH2 split, a new N- as well as a C-terminus is generated. Variants (7)-(9) are based on the Fv-only and Fab-like format constructs (1)-(3) now fused to an IgG Fc-domain generating tetravalent bispecific antibody variants. Variants (11) and (12) are bivalent bispecific antibodies. On one side of the antibody the CH1 and CL domains are replaced by the split-CH2 domain. Since only one light chain is used, this construct design avoids the light chain mispairing problem. Heterodimerization of the heavy chains is achieved by applying knob-into-hole-mutations. (A) Diabody split CH2 tetravalent bispecific antibody. (B) DVD split CH2 tetravalent bispecific antibody. (C) CODV split CH2 tetravalent bispecific antibody. (D, E) Spit CH2 bivalent bispecific antibody.

FIG. 6: SDS-PAGE of protein variants (8)-(12) analyzed under non-reducing and reducing conditions. All proteins were purified via the Fc-domain on a protein A matrix. Two protein chains are required for variants (8)-(10) whereas four protein chains are required for variants (11) and (12). From the staining intensities of the protein bands in the reduced form it can roughly be estimated that the protein bands are present in equimolar amounts. Note, that for (11) and (12) the two heavy chains have roughly the same molecular weight and are not resolved into distinct bands on the SDS-PAGE.

FIG. 7: Mass spectrometry analysis of protein variant (1). (A) Deglycosylated, non-reducing conditions. (B) Deglycosylated, reducing conditions.

FIG. 8: Mass spectrometry analysis of protein variant (2). (A) Deglycosylated, non-reducing conditions. (B) Deglycosylated, reducing conditions.

FIG. 9: Mass spectrometry analysis of protein variant (3). (A) Deglycosylated, non-reducing conditions. (B) Deglycosylated, reducing conditions.

FIG. 10: Mass spectrometry analysis of protein variant (4). (A) Deglycosylated, non-reducing conditions. (B) Deglycosylated, reducing conditions.

FIG. 11: Mass spectrometry analysis of protein variant (5). (A) Deglycosylated, non-reducing conditions. (B) Deglycosylated, reducing conditions.

FIG. 12: Mass spectrometry analysis of protein variant (6). (A) Deglycosylated, non-reducing conditions. (B) Deglycosylated, reducing conditions.

FIG. 13: Mass spectrometry analysis of protein variant (7). (A) Deglycosylated, non-reducing conditions. (B) Deglycosylated, reducing conditions.

FIG. 14: Mass spectrometry analysis of protein variant (8). (A) Deglycosylated, non-reducing conditions. (B) Deglycosylated, reducing conditions.

FIG. 15: Mass spectrometry analysis of protein variant (9). (A) Deglycosylated, non-reducing conditions. (B) Deglycosylated, reducing conditions.

FIG. 16: Mass spectrometry analysis of protein variant (10). (A) Deglycosylated, non-reducing conditions. (B) Deglycosylated, reducing conditions.

FIG. 17: Mass spectrometry analysis of protein variant (11). (A) Deglycosylated, non-reducing conditions. (B) Deglycosylated, reducing conditions.

FIG. 18: Mass spectrometry analysis of protein variant (12). (A) Deglycosylated, non-reducing conditions. (B) Deglycosylated, reducing conditions.

FIG. 19: Biacore measurement of protein variant (4) to the human FcRn which was immobilized on the chip.

FIG. 20: SDS-PAGE of protein variants (13)-(16) analyzed under non-reducing and reducing conditions. Variants (13)-(16) require each two protein chains. Note that for all four variants comprise potential N-glycosylation sites (variant (13): N-terminal split CH2 domain, position 144; variant (14): C-terminal split CH2 domain, position 255; variant (15): N- and C-terminal split CH3 domain, positions 252 and 275, respectively; variant (16) N-terminal split CH2 domain, Pos. 146). These modifications might be the reason for the fuzzy appearance of some protein bands in the SDS-PAGE, especially for protein variants (15) and (16).

FIG. 21: Mass spectrometry analysis of protein variant (13). (A) Deglycosylated, non-reducing conditions. (B) Deglycosylated, reducing conditions.

FIG. 22: Mass spectrometry analysis of protein variant (14). (A) Deglycosylated, non-reducing conditions. (B) Deglycosylated, reducing conditions.

FIG. 23: Stability measurements of protein variants (13)-(16). Tryptophan fluorescence measurement allows monitoring the melting of a protein. The measured fluorescence ration F350 nm/F330 nm represents the melting curves of the proteins. The calculated Tₘ (Tab.2) corresponds to the maximum of the first derivative of the F350/F330 curve.

### EXAMPLE SECTION

### Material and Methods

### Protein Expression and Purification

DNA coding for the desired amino acid sequences were synthesized (Thermofisher, Geneart) and cloned into an expression vector under a CMV promoter and a signal sequence required for secretion of the proteins into the cell culture medium. Protein expression was done by transient transfection of FreeStyle HEK293-F-cells (Thermo Fisher Scientific). Cells were cultivated in non-baffled shake flasks (Corning) at 110 rpm, 37° C. and 8% C CO₂. Transfection was done when the cell density reached 1.2×106 cells/mL. DNA was mixed at a ratio of 1:3 with Polyethyleneimine in Optimem I-Medium (Thermo Fisher Scientific). After 20 min incubation at room temperature the transfection mix was added to the cell culture. Cells were further cultivated in FreeStyle F17 medium complemented with 6 mM glutamine for 6 days. Cells were removed from the culture broth by centrifugation (30 min at 4.500 g, 4° C.) and the supernatant was cleared by 0.22 μm sterile filtration. Protein variants (1)-(7) as well as the CH2 domain wt-only construct contain a consecutive stretch of six histidine-residues (His-tag). Protein purification was done by immobilized metal-ion affinity chromatography (IMAC) with a complete His-tag purification column (Roche) on a NGC Discover 100 Pro system (Biorad). The column was equilibrated with 50 mM Tris, 500 mM NaCl, 10 mM histidine. Before loading the culture supernatant, the pH was adjusted to 8.0 by adding 50 mL per L of a 1 M Tris, pH8.0 solution. In addition, 5 mL of a 2 M imidazole, pH 7.5 solution were added per L. The column was washed with equilibration buffer and proteins were eluted with a 35 CV gradient to 50 mM Tris, 300 mM NaCl, 500 mM imidazole. Fractions were analyzed by SDS-PAGE, corresponding fractions were pooled, concentrated by centrifugation (Vivaspin 20) and loaded on a Superdex 200 pg 16/60 (GE Healthcare) gelfiltration column equilibrated in phosphate buffered saline (PBS, Gibco). Fractions containing the desired protein were pooled and concentrated (Vivaspin 20). Protein variants (8)-(12) do not have a His-tag but rather a Fc-domain. For these variants capture was done on a HiTrap Protein A column (GE Healthcare). The column was equilibrated with PBS. After loading the cleared supernatant, the column was washed with PBS (Gibco) and 0.1 M citrate pH 6.0. Proteins were eluted with 0.1 M citrate, pH 3.0 buffer. Eluted fractions were neutralized by adding 10% 1 M Tris pH 8.5. Further purification was done using a Superdex 200 pg 16/69 (GE Healthcare) gelfiltration column equilibrated in PBS (Gibco). Protein concentrations were determined by measuring the absorption at 280 nm using a NanoDrop NT1000 spectrophotometer (Thermo Fisher Scientific). SDS-PAGE analysis was done using 4-12% BisTris gels with IVIES-buffer as running buffer (Invitrogen). For reduced samples, 0.1 M DTT were added to sample buffer (LDS sample buffer, invitroge) and samples were incubated for 5 min at 99° C. Separation was done with constant 200 V for 45 min. The BenchMark protein ladder was used as marker (invitrogen). After running, gels were stained with Coomassie blue (InstantBlue, Expedeon).

### Thermal Stability Analysis

Thermal stability analysis was done with a Prometheus NT Flex device (NanoTemper Technologies) using the nanoDSF technology. The device is equipped with Aggregation Optics, that allows collecting scattering information simultaneously with fluorescence measurements. Analysis was done in the range from 20° C. to 95° C. with a thermal ramp of 1° C./min following the instructions of the manufacturer. All protein samples were dissolved in PBS and protein concentration was adjusted to 0.5 mg/mL. Measurements were done in duplicates. Data analysis was done using the software PR ThermControl V.2.1 (NanoTemper Technologies).

### MS-Analysis

Protein integrity was analyzed by LC-MS. Protein samples were deglycosylated with 12.5 μg of protein diluted to 0.5 mg/ml in ddH2O containing PNGaseF (1:50 v/v) (glycerol free, New England Biolabs) at 37° C. for 15 hours. The LC-MS analysis was done on an Agilent 6540 Ultra High Definition (UHD) Q-TOF equipped with a dual ESI interface and an Agilent 1290/1260 Infinity LC System. Reversed phase (RP) chromatography was done using a PLRP-S 1000A 5 μm, 50×2.1 mm (Agilent) with a guard column PLRP-S 300A 5 μm, 3×5 mm (Agilent) at 200 μL/min and 80° C. column temperature. Eluents were buffer A containing LC water and 0.1% formic acid as well as buffer B containing 90% acetonitrile, 10% LC water and 0.1% formic acid. 1 μg of protein was injected onto the column and eluted using a linear gradient from 0 to 17 minutes with increasing acetonitrile concentration. Data were analyzed using MassHunter Bioconfirm B.06 (Agilent). Molecular masses were calculated based on the amino acid sequences of the proteins using GPMAW software version 10.32 (Lighthouse Data, Denmark).

### Affinity Determinations

Binding of antigens to the antibody constructs was measured using surface plasmon resonance (SPR) on a BIAcore 3000 instrument (GE Healthcare) with HBS-EP buffer (GE Healthcare). As antigens human IL4 (IL004, Millipore) and human IL13 (IL012, Millipore) were used. The capture antibody (human antibody capture kit, His capture kit, Fab capture kit, GE Life Sciences) was immobilized via primary amine groups (11000 RU) on a research grade CMS chip (GE Life Sciences) using standard procedures. The ligands were captured at a flow rate of 10 μl/min with an adjusted RU value that resulted in maximal analyte binding of 30 RU. The antigens human IL4 and human IL13 were used as analytes and injected for 240 sec with a dissociation time of 300 sec at a flow rate of 30 μL/min. IL4 and IL13 were used in a dilution series of 0.1 nM to 3 nM and 0.8 nM to 25 nM, respectively. Chip surfaces were regenerated with 2 min injects of the regeneration buffer provided with the capture kit. Sensorgrams were double referenced with a blank chip surface and HBS-EP buffer blanks. Recombinant human neonatal Fc-receptor (FcRn) protein was immobilized via primary amine groups (200 RU) in the sample flow cell compartment of a research grade CMS chip (GE Life Sciences) using standard procedures. The reference flow cell compartment was activated and deactivated without FcRn immobilization to generate a blank chip surface. For the analysis an assay buffer with pH 6.0 was used (150 mM NaCl, 20 mM Na-phosphate, 0.05% surfactant P20, pH6.0). The antibody was used as analyte at 800 nM dilution in assay buffer and injected over reference and sample flow cells for 240 sec with a dissociation time of 300 sec at a flow rate of 30 μL/min. Chip surfaces were regenerated with 2 min injects of HBS-EP buffer at 30 μl/min. Sensorgrams were double referenced with a the blank chip surface and HBS-EP buffer blanks. All data analysis was done using the BIAevaluation software v4.1.

### Results

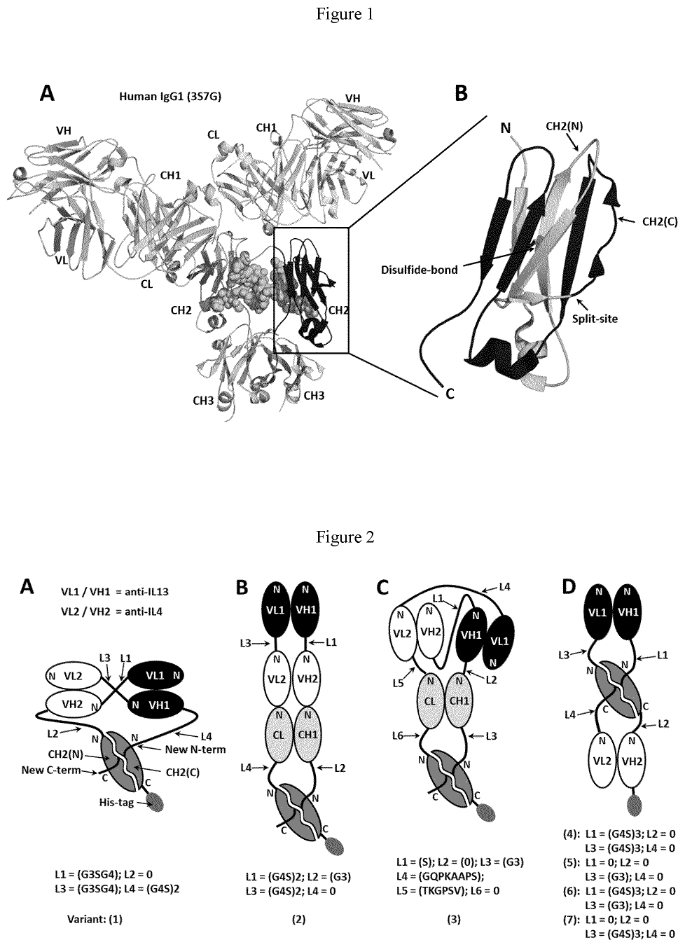

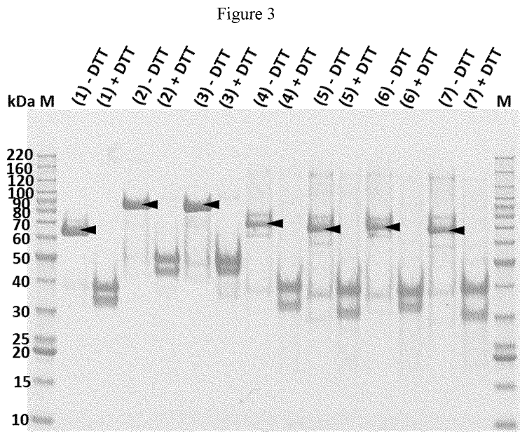

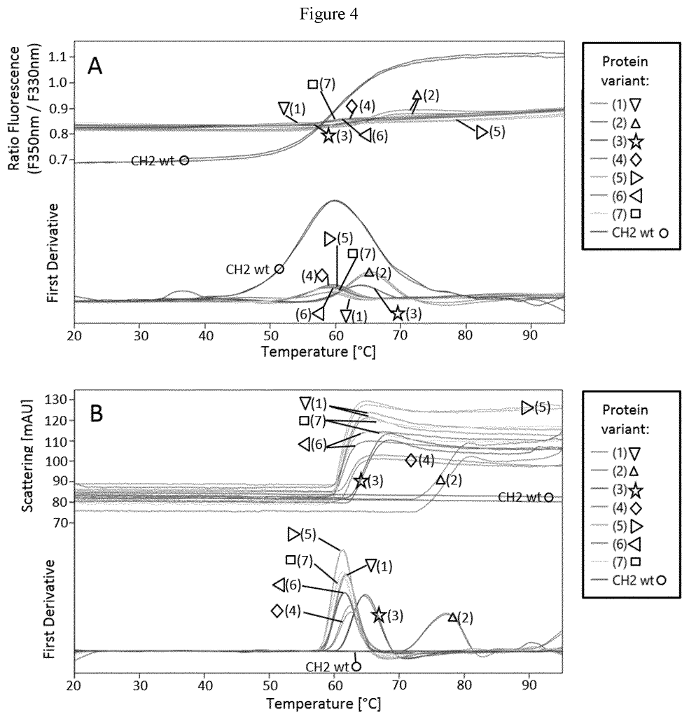

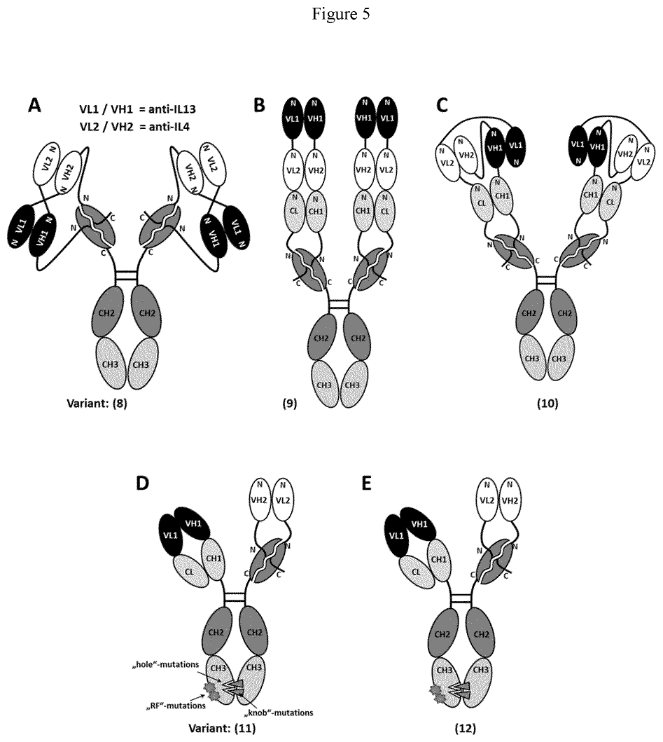

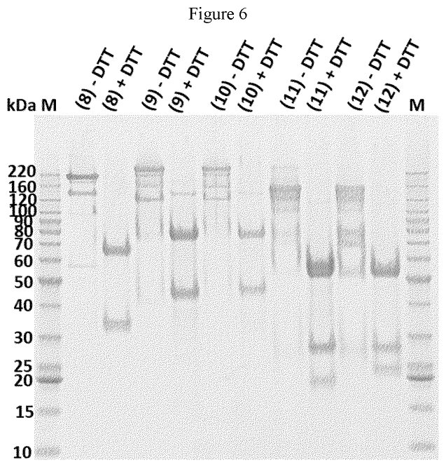

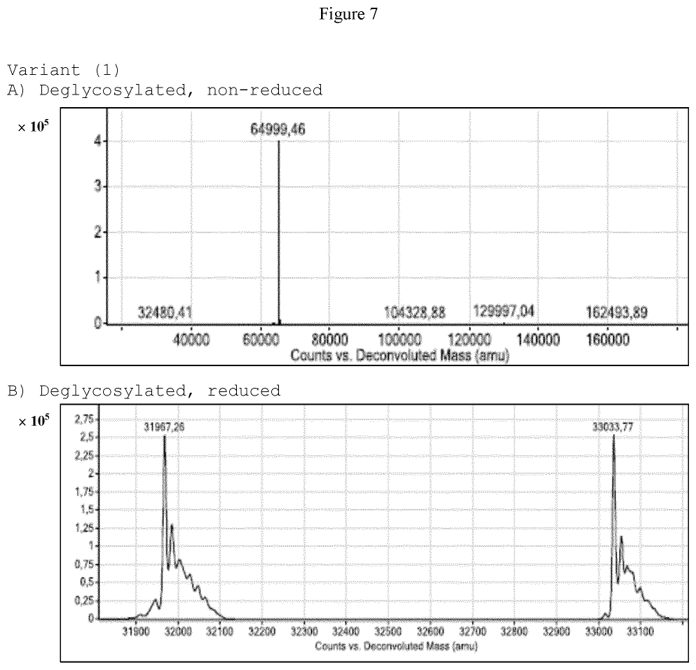

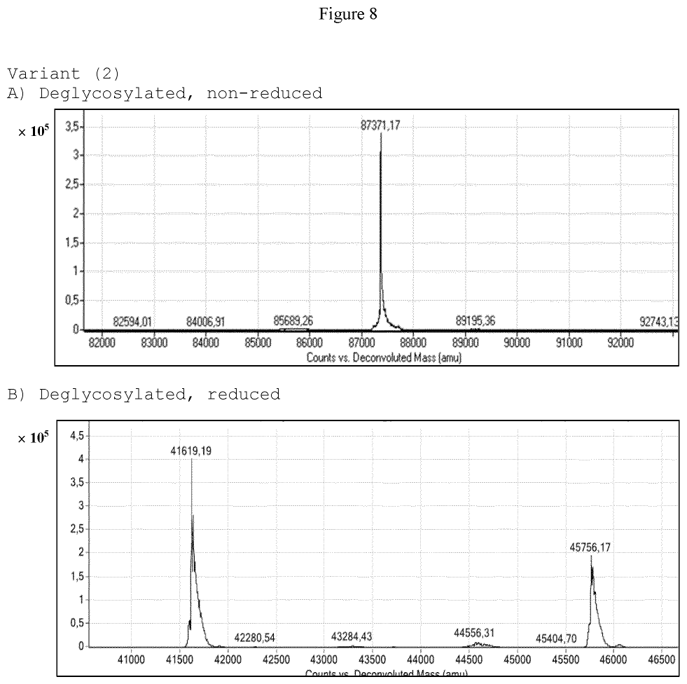

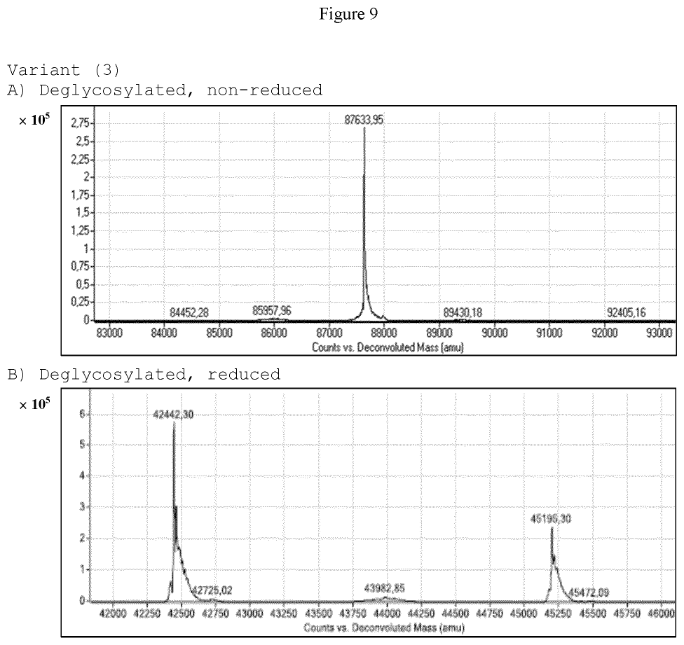

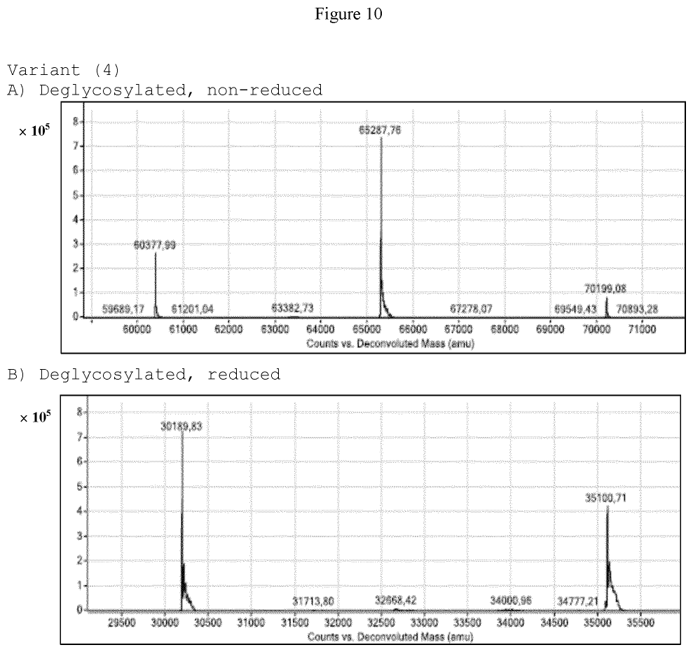

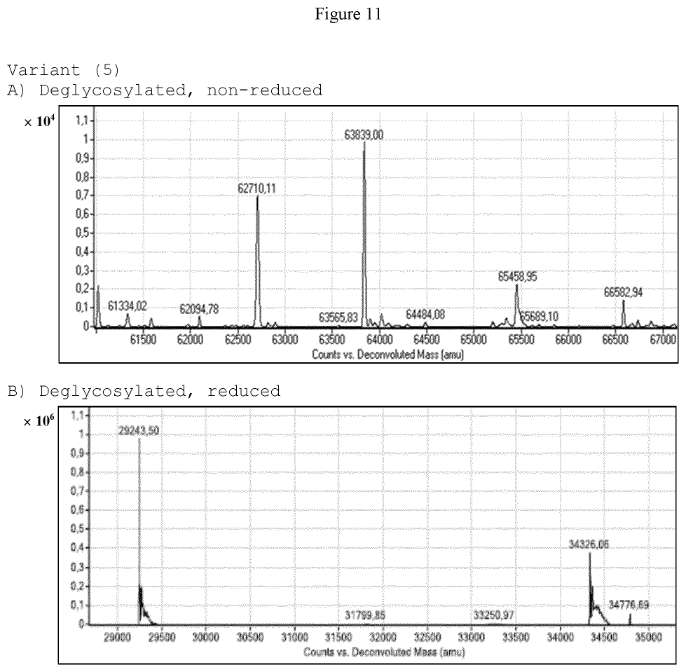

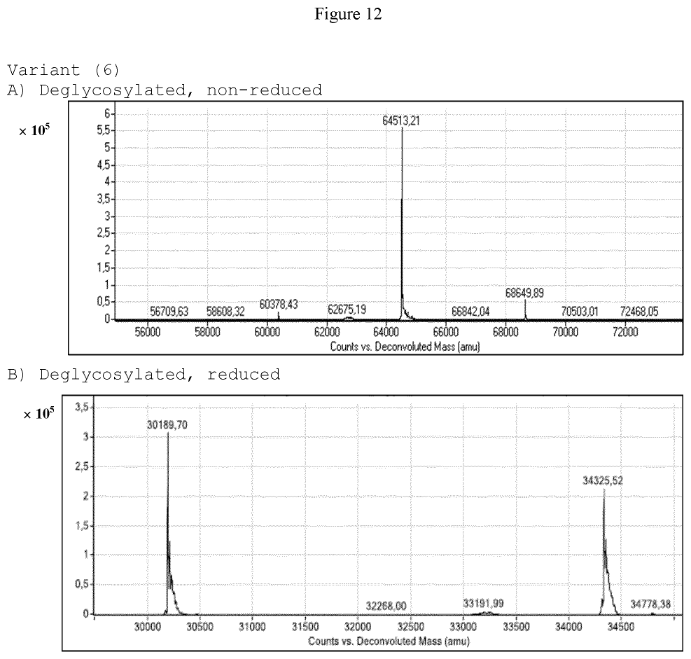

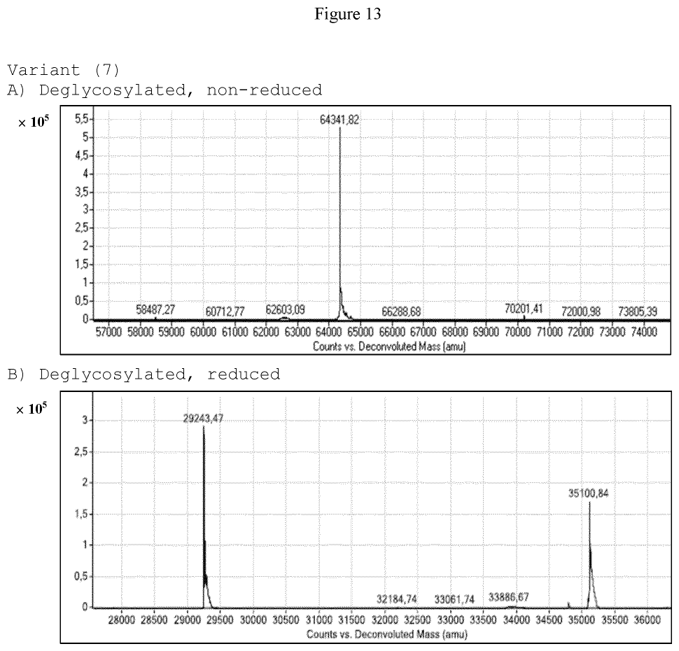

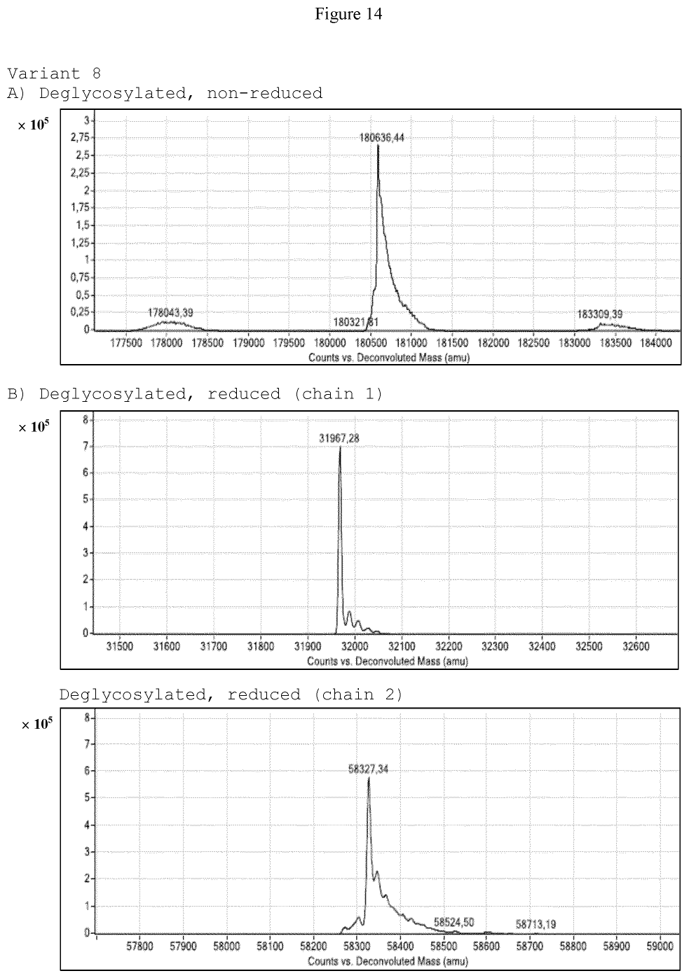

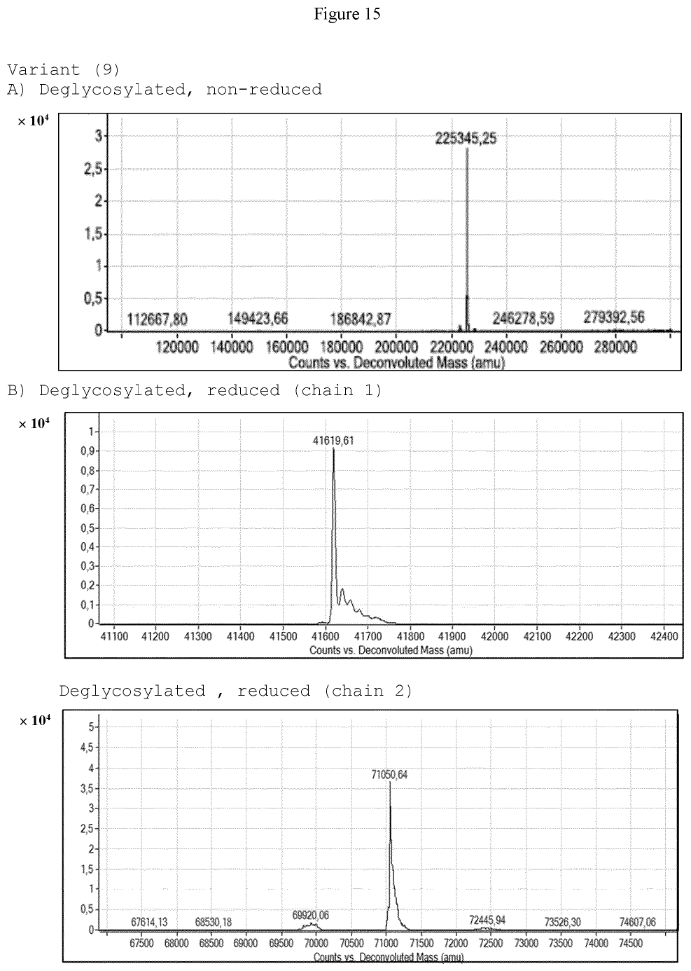

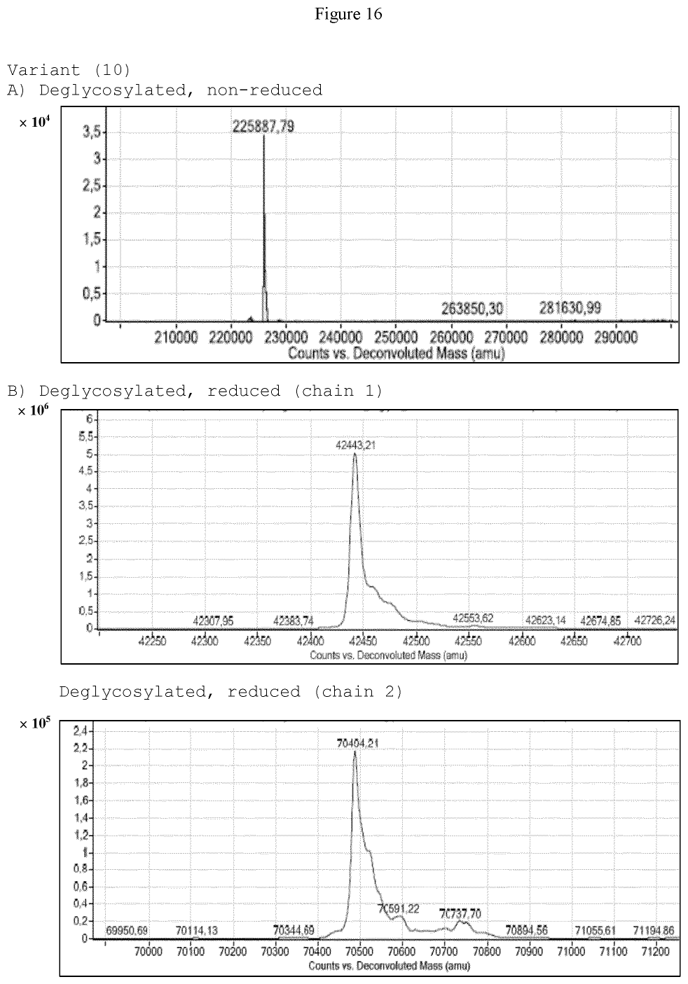

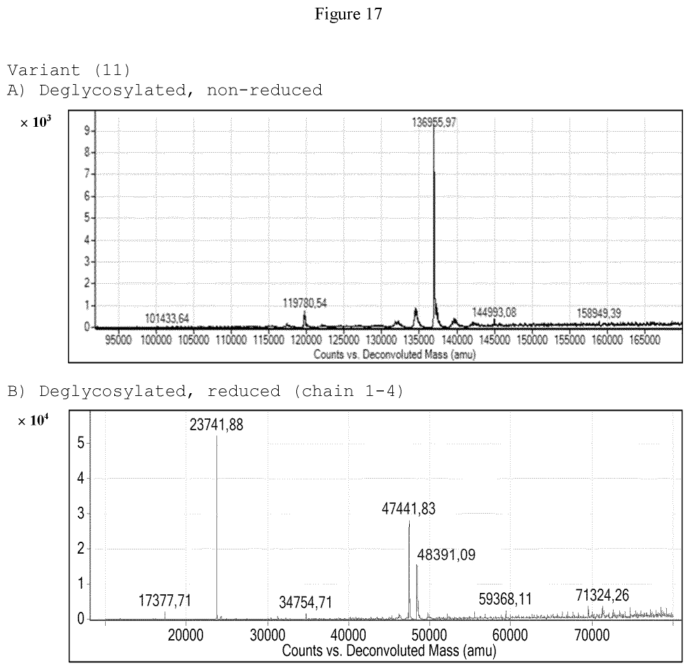

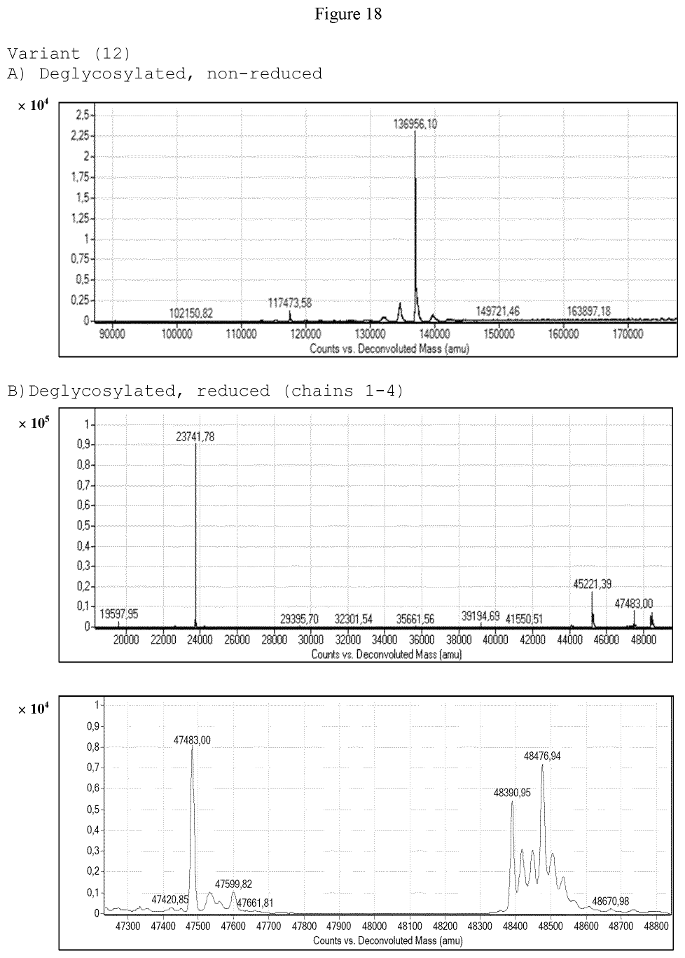

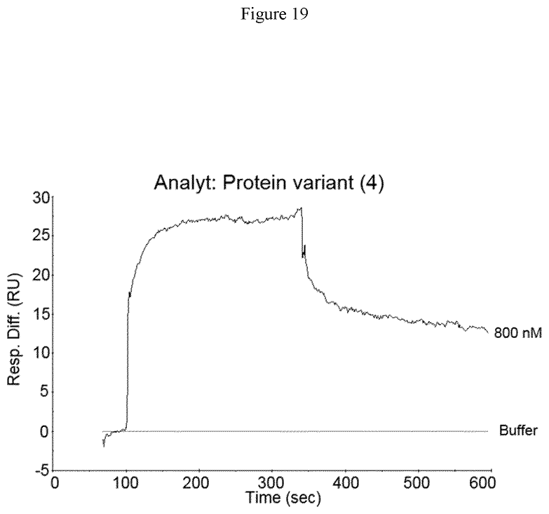

It was envisaged to split the IgG1 CH2 domain in two parts with roughly the same size in a similar manner as it had been described for the split-ubiquitin-system. This is in contrast to the split GFP or β-galactosidase approach, where only small parts of the entire protein are sufficient to restore functionality. The structural basis for assessing the split site is shown in FIG. 1. As first attempts to evaluate the reassembly of the split CH2 domain the inventors added two different variable domains with or without the CH1 and CL domains thus creating different formats (FIG. 2). The two different variable domains directed against interleukins IL4 and IL13 are those of a bispecific antibody currently in clinical development and have been described in detail before (Steinmetz et al.; Mabs 2016; 8:867-878). The sequence of the N-terminal part of the split CH2 domain as used in this report consists of 52 amino acids (5.7 kDa) and is characterized by SEQ ID NO: 30. The sequence of the C-terminal part of the split CH2 domain as used in this report consists of 58 amino acids (6.7 kDa) and is characterized by SEQ ID NO: 31. Upon re-assembly of the split CH2 domain, the disulfide-bond between the two parts was expected to form and hence the two protein chains should be covalently connected. Since only one protein chain was fused to a tag for purification only the heterodimeric molecules should be purified. The connection of the two chains by a disulfilde-bond could easily be monitored using SDS-PAGE running under reduced compared to non-reduced conditions (FIG. 3). As apparent from these results, the split CH2 domain was reconstituted and the disulfide-bond connecting the two split CH2 half was formed. Upon reduction of the protein samples, the only disulfide-bond connecting the two protein chains is broken. This disulfide-bond is formed between the split CH2 domain parts. The observed main protein bands migrated in the SDS-PAGE at their predicted size. To further assess whether the produced proteins contain a reassembled CH2 domain the inventors analyzed the protein stability using differential scanning fluorimetry (DSF) technology (FIG. 4, Tab.2). This technique monitors the intrinsic fluorescence of tryptophan residues within a protein. Tryptophan fluorescence is highly sensitive to its immediate environment. Upon protein conformational changes, for example during denaturation, the fluorescence emission maxima is shifted. The inventors anticipate that a distinct melting temperature (Tₘ) of the protein can be measured if the split-CH2 domain is re-assembled. Simultaneously to the fluorescence measurements light scattering data were recorded that allow calculation of the onset of protein aggregation (T_{agg}). The obtained Tₘ and T_{agg} data are given in Table 2. The measured Tₘ values are in the range of ˜60° C. or slightly higher and in the range of the Tₘ of the CH2-domain wt construct. These data suggest that the split CH2 domain is reassembled. Protein variants (2) and (3) have a slightly higher Tₘ and protein variant (2) has a T_{agg} that is ˜12° C. higher. The elevated Tₘ and T_{agg} are probably due to the presence of the CL and the CH1 domains which further stabilize the protein. Next, the inventors assessed whether it is possible to express the split CH2 domain only and purify the reassembled CH2-domain. However, the inventors could not detect any expression of the split CH2 domain. Probably, a folded fusion partner is required for proper expression and reconstitution of the split CH2 domain. As the expression of the split CH2 domain worked well with the Fv- and Fab only format, the inventors next evaluated the expression of these constructs when incorporated within an IgG-like architecture (FIG. 5A-C). In addition, the inventors used the split CH2-domain to address the light chain pairing problem in a bispecific heterodimeric IgG-like format (FIG. 5 D,E). All variants could be expressed and purified (FIG. 6). All proteins containing the re-assembled split CH2 domain were analyzed by mass spectroscopy under oxidizing and reducing conditions. The presence of the anticipated species and the corresponding protein chains could be demonstrated (FIG. 7-18). The inventors' focus was to show that a split CH2 domain can be in principal incorporated into a larger molecule and is able to reassemble in such an architecture. Correct assembly of the target molecules was demonstrated by evaluating the binding properties to the corresponding antigens. This was checked by surface plasmon resonance (SPR)-measurements (Tab.3, Tab.7-20). Structural changes below the Fv domains might lead to subtle changes in the Fv domains, and as a consequence thereof loss in binding affinity could occur. However, only very minor differences in binding affinity between the various constructs compared to the reference molecule, a bispecific anti-IL4-anti-IL13 CODV-IgG (variant 13) (Steinmetz et al.; Mabs 2016; 8:867-87), were detected. In addition, binding of the reconstituted CH2 domain to human FcRn was analyzed by Biacore analysis. FcRn was immobilized on the chip and variant (4) was used as analyte. The measurement shows binding of variant (4) to the FcRn (FIG. 19).

In contrast to variants (1)-(12), which are all based on the IgG1 split CH2 domain, variants (13)-(16) comprise CH domains of other antibody classes. Variants (13)-(16) have the diabody split CH2 format as used in variant (1), but the IgG1 split CH2 domain is exchanged for one the following split CH domains: variant (13): split IgA CH2; variant (14): split IgD CH2; variant (15): split IgE CH3; and variant (16): split IgE CH2. Tab. 4 indicates the sequences of the variants.

Experimental procedures were performed as described for variants (1)-(12) above.

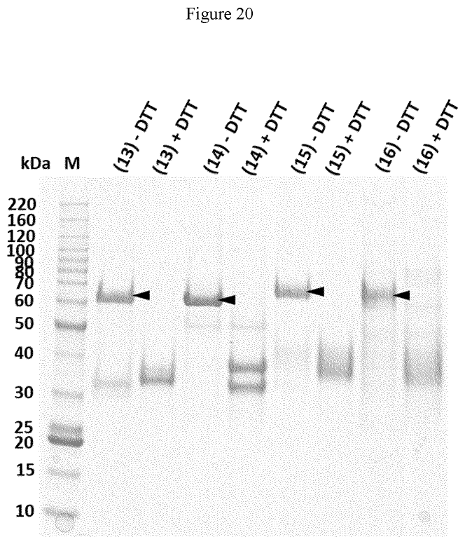

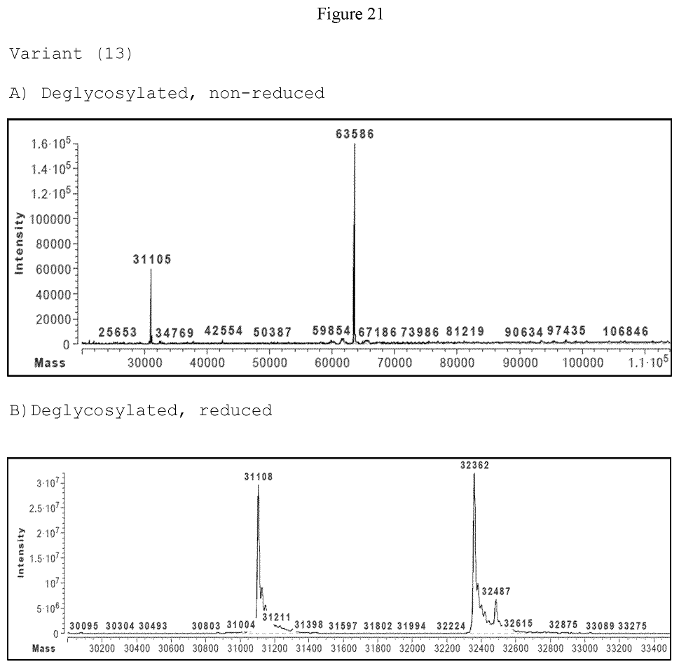

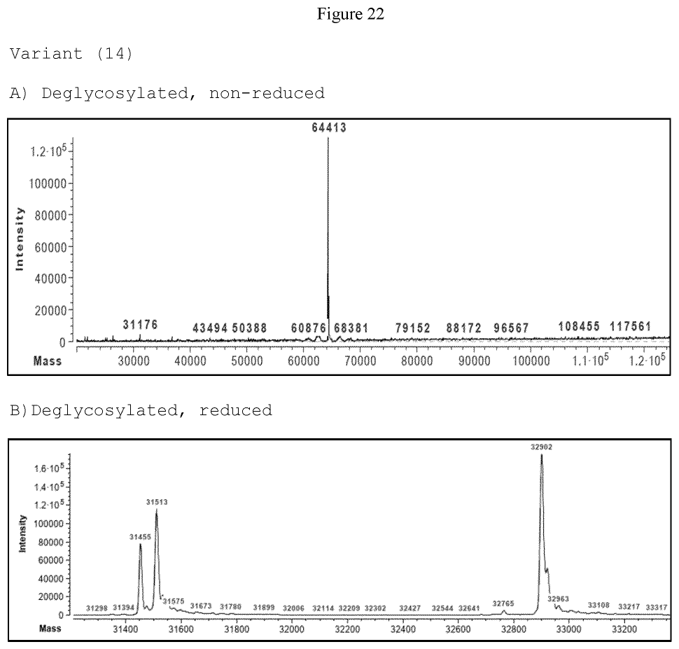

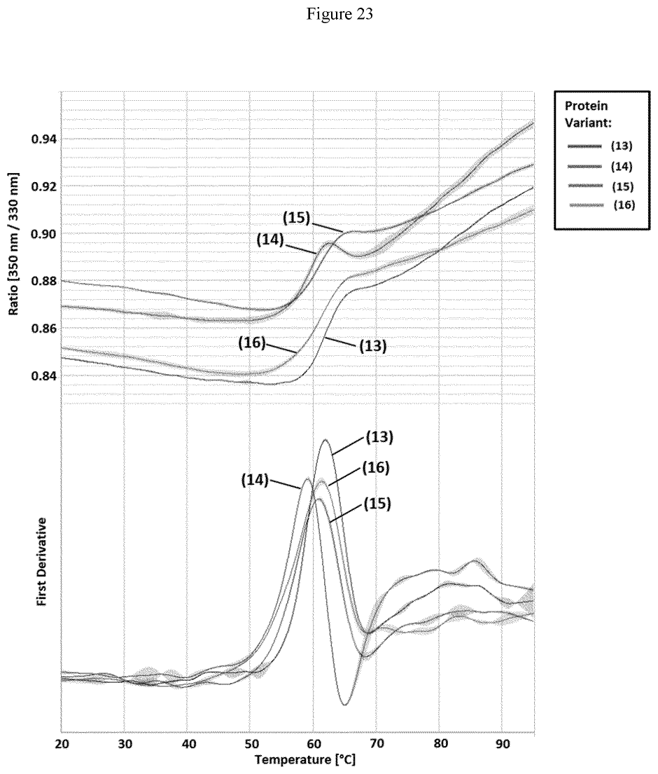

SDS-PAGE analysis of variants (13)-(16) (FIG. 20) demonstrates that all proteins can be produced and assemble. Thermal stability measurements show a distinct melting point in all cases (FIG. 23, Tab. 2). The obtained melting temperature is comparable to the wild-type CH2 domain indicating that the proteins are correctly folded. Variants (13) and (14) were also analyzed by mass spectroscopy (FIGS. 21 and 22, Tab. 5 and 6). In summary, the results for variants (13)-(16) demonstrate that splitting and re-assembly is not restricted to the IgG1 CH2 domain.

TABLE 2
stability of the reassembled CH2 domain
as measured by tryptophan fluorescence.

| | | Tₘ | T_{agg} |
|---|---|---|---|
| Variant | Description | (average) | (average) |
| (1) | Diabody-added Split CH2 | 59.01 | 59.79 |
| (2) | DVD-Fab Split CH2 | 65.33 | 72.09 |
| (3) | CODV-Fab Split CH2 | 63.88 | 61.87 |
| (4) | Diabody Split CH2 (included) (“Splite”) | 59.51 | 59.94 |
| (5) | Diabody Split CH2 (included) (“Splite”) | 59.46 | 59.25 |
| (6) | Diabody Split CH2 (included) (“Splite”) | 58.97 | 59.08 |
| (7) | Diabody Split CH2 (included) (“Splite”) | 58.43 | 58.88 |
| (13) | Diabody-added Split IgA CH2 | 61.95 | 63.50 |
| (14) | Diabody-added Split IgD CH2 | 59.01 | 63.43 |
| (15) | Diabody-added Split IgE CH3 | 60.85 | n.d. |
| (16) | Diabody-added Split IgE CH2 | 61.33 | n.d. |
| | CH2-domain (wt) | 59.94 | n.d. |

Tₘ: melting temperature.
T_{agg}: onset of protein aggregation.
Average of two measurements.
n.d.: not determined.

TABLE 3
Antigen affinities for the different protein
variants as determined by SPR.

| Variant | Description | K_{D} (IL4) [M] | K_{D} (IL13) [M] |
|---|---|---|---|
| | CODV-IgG | 2.72 × 10⁻¹² | 2.45 × 10⁻¹¹ |
| (1) | Diabody-added Split CH2 | 1.60 × 10⁻¹¹ | 1.01 × 10⁻¹⁰ |
| (2) | DVD-Fab Split CH2 | 3.0 × 10⁻¹¹ | 1.05 × 10⁻¹⁰ |
| (3) | CODV-Fab Split CH2 | 5.48 × 10⁻¹² | 6.7 × 10⁻¹¹ |
| (4) | Diabody Split CH2 (included) | 6.48 × 10⁻¹¹ | 2.74 × 10⁻¹¹ |
| | (“Splite”) | | |
| (5) | Diabody Split CH2 (included) | 4.03 × 10⁻¹¹ | 1.46 × 10⁻¹¹ |
| | (“Splite”) | | |
| (6) | Diabody Split CH2 (included) | 8.78 × 10⁻¹¹ | 2.57 × 10⁻¹¹ |
| | (“Splite”) | | |
| (7) | Diabody Split CH2 (included) | 6.85 × 10⁻¹¹ | 4.31 × 10⁻¹¹ |
| | (“Splite”) | | |
| (8) | Diabody Split CH2 tetravalent | 1.43 × 10⁻¹² | 4.36 × 10⁻¹¹ |
| | bispecific antibody | | |
| (9) | DVD-Split CH2 tetravalent | 6.09 × 10⁻¹¹ | 3.64 × 10⁻¹¹ |
| | bispecific antibody | | |
| (10) | CODV-Split CH2 tetravalent | 2.22 × 10⁻¹³ | 3.31 × 10⁻¹¹ |
| | bispecific antibody | | |
| (11) | Split CH2 bivalent bispecific | 1.30 × 10⁻¹² | 1.96 × 10⁻¹¹ |
| | antibody | | |
| (12) | Split CH2 bivalent bispecific | 9.22 × 10⁻¹³ | 8.24 × 10⁻¹² |
| | antibody | | |

TABLE 4
Polypeptide sequences of variants (1)-(16)

| Variant | Chain 1 | Chain 2 | Chain 3 | Chain 4 |
|---|---|---|---|---|
| 1 | SEQ ID 11 | SEQ ID 12 | — | — |
| 2 | SEQ ID 13 | SEQ ID 14 | — | — |
| 3 | SEQ ID 15 | SEQ ID 16 | — | — |
| 4 | SEQ ID 17 | SEQ ID 18 | — | — |
| 5 | SEQ ID 19 | SEQ ID 20 | — | — |
| 6 | SEQ ID 17 | SEQ ID 20 | — | — |
| 7 | SEQ ID 19 | SEQ ID1 8 | — | — |
| 8 | SEQ ID 11 | SEQ ID 21 | — | — |
| 9 | SEQ ID 13 | SEQ ID 22 | — | — |
| 10 | SEQ ID 15 | SEQ ID 23 | — | — |
| 11 | SEQ ID 24 | SEQ ID 27 | SEQ ID 26 | SEQ ID 25 |
| 12 | SEQ ID 24 | SEQ ID 27 | SEQ ID 28 | SEQ ID 29 |
| 13 | SED ID 41 | SED ID 42 | — | — |
| 14 | SED ID 43 | SED ID 44 | — | — |
| 15 | SED ID 45 | SED ID 46 | — | — |
| 16 | SED ID 47 | SED ID 48 | — | — |

TABLE 5
Intact mass, deglycosylated, non-reduced

| | Calculated | Measured |
|---|---|---|
| Variant | MW (Da) | MW (Da) |
| 1 | 64996.68 | 64999.46 |
| 2 | 87366.46 | 87371.17 |
| 3 | 87646.04 | 87633.95 |
| 4 | 65283.94 | 65287.76 |
| 5 | 63563.40 | 63839.00 |
| 6 | 64509.25 | 64513.21 |
| 7 | 64338.09 | 64341.82 |
| 8 | 180567.66 | 180636.44 |
| 9 | 225309.20 | 225345.25 |
| 10 | 225868.34 | 225887.79 |
| 11 | 136953.87 | 136955.97 |
| 12 | 136953.87 | 136956.10 |
| 13 | 63461.68 | 63586.0 |
| 14 | 64410.00 | 64413.0 |

Molecular weight were calculated with GPMAW (Vers. 10.32; Lighthouse Data, Denmark) (average mass values, all cysteines are assumed to form disulfid-bridges).

TABLE 6
Intact mass, deglycosylated, reduced

| | Chain 1 | Chain 1 | Chain 2 | Chain 2 | Chain 3 | Chain 3 | Chain 4 | Chain 4 |
|---|---|---|---|---|---|---|---|---|
| | Calc. | Meas | Calc. | Meas. | Calc | Meas. | Calc | Meas |
| Variant | MW (Da) | MW (Da) | MW (Da) | MW (Da) | MW (Da) | MW (Da) | MW (Da) | MW (Da) |
| 1 | 31970.20 | 31967.26 | 33035.57 | 33033.77 | | | | |
| 2 | 41623.89 | 41619.19 | 45758.69 | 45756.17 | | | | |
| 3 | 42446.97 | 42442.30 | 45215.20 | 45195.30 | | | | |
| 4 | 30192.28 | 30189.83 | 35101.73 | 35100.71 | | | | |
| 5 | 29246.44 | 29243.50 | 34327.04 | 34326.06 | | | | |
| 6 | 30192.28 | 30189.70 | 34327.04 | 34325.52 | | | | |
| 7 | 29246.44 | 29243.47 | 35101.73 | 35100.84 | | | | |
| 8 | 31970.20 | 31967.28 | 58329.76 | 58327.34 | | | | |
| 9 | 41623.89 | 41619.61 | 71052.88 | 71050.64 | | | | |
| 10 | 42446.97 | 42443.21 | 70509.39 | 70404.21 | | | | |
| 11 | 48396.95 | 48391.09 | 23746.05 | 23741.88 | 47461.70 | 47441.83 | 17379.40 | 17377.71 |
| 12 | 48396.95 | 48390.95 | 23746.05 | 23741.78 | 45224.38 | 45221.39 | 19613.70 | 19597.95 |
| 13 | 31110.19 | 31108.0 | 32364.59 | 32362.0 | | | | |
| 14 | 31515.78 | 31513.0 | 32904.29 | 32902.0 | | | | |

Molecular weights were calculated with GPMAW (Vers. 10.32; Lighthouse Data, Denmark) (average mass values, all cysteines are assumed to be reduced (SH))

### Biacore-Measurements

TABLE 7
Biacore measurement of protein variant (1) immobilized
with the aid of the His-capture kit.

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 187 | IL4_3.125 nM | 7.32E+07 | 1.17E−03 | 37 | 6.24E+10 | 1.60E−11 | 1.760 |
| 185 | IL13_25 nM | 1.99E+06 | 2.00E−04 | 34 | 9.93E+09 | 1.01E−10 | 0.416 |

TABLE 8
Biacore measurement of protein variant (2) immobilized
with the aid of the His-capture kit.

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 291 | IL4_3.125 nM | 1.42E+07 | 4.26E−04 | 46 | 3.33E+10 | 3.00E−11 | 0.997 |
| 291 | IL13_25 nM | 2.03E+06 | 2.14E−04 | 40 | 9.49E+09 | 1.05E−10 | 0.395 |

TABLE 9
Biacore measurement of protein variant (2) immobilized
with the aid of the Fab-capture kit

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 187 | IL4_3.125 nM | 1.48E+07 | 2.59E−04 | 38 | 5.73E+10 | 1.75E−11 | 1.610 |
| 187 | IL13_25 nM | 1.99E+06 | 2.12E−04 | 33 | 9.39E+09 | 1.06E−10 | 0.713 |

TABLE 109
Biacore measurement of protein variant (3) immobilized
with the aid of the Fab-capture kit.

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 185 | IL4_3.125 nM | 8.26E+07 | 4.52E−04 | 43 | 1.83E+11 | 5.48E−12 | 1.310 |
| 185 | IL13_25 nM | 2.03E+06 | 1.36E−04 | 33 | 1.49E+10 | 6.70E−11 | 1.370 |

TABLE 11
Biacore measurement of protein variant (4) immobilized
with the aid of the His-capture kit.

| | | | | Rmax | | |
|---|---|---|---|---|---|---|
| RU 2nd Ab | Analyte | ka (1/Ms) | kd (1/s) | (RU) | KD (M) | Chi2 |
| 187 | IL13_25 nM | 5.82E+06 | 1.60E−04 | 22 | 2.74E−11 | 0.332 |
| 187 | IL4_3.125 nM | 3.46E+06 | 2.24E−04 | 26 | 6.48E−11 | 0.529 |

TABLE 12
Biacore measurement of protein variant (5) immobilized
with the aid of the His-capture kit.

| | | | | Rmax | | |
|---|---|---|---|---|---|---|
| RU 2nd Ab | Analyte | ka (1/Ms) | kd (1/s) | (RU) | KD (M) | Chi2 |
| 331 | IL13_25 nM | 1.29E+07 | 1.88E−04 | 10 | 1.46E−11 | 0.223 |
| 331 | IL4_3.125 nM | 4.10E+06 | 1.65E−04 | 11 | 4.03E−11 | 0.141 |

TABLE 13
Biacore measurement of protein variant (6) immobilized
with the aid of the His-capture kit.

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 195 | IL13_25 nM | 4.07E+06 | 1.05E−04 | 20 | 3.89E+10 | 2.57E−11 | 0.277 |
| 195 | IL4_3.125 nM | 2.97E+06 | 2.61E−04 | 30 | 1.14E+10 | 8.78E−11 | 0.328 |

TABLE 14
Biacore measurement of protein variant (7) immobilized
with the aid of the His-capture kit.

| | | | | Rmax | | | |
|---|---|---|---|---|---|---|---|
| RU 2nd Ab | Analyte | ka (1/Ms) | kd (1/s) | (RU) | KA (1/M) | KD (M) | Chi2 |
| 188 | IL13_25 nM | 4.14E+06 | 1.78E−04 | 21 | 2.32E+10 | 4.31E−11 | 0.326 |
| 188 | IL4_3.125 nM | 3.38E+06 | 2.32E−04 | 28 | 1.46E+10 | 6.85E−11 | 0.29 |

TABLE 15
Biacore measurement of protein variant (8) immobilized
with the aid of the human antibody capture kit.

| | | | | Rmax | | |
|---|---|---|---|---|---|---|
| RU 2nd Ab | Analyte | ka (1/Ms) | kd (1/s) | (RU) | KD (M) | Chi2 |
| 229 | IL13_25 nM | 3.46E+06 | 1.51E−04 | 35 | 4.36E−11 | 0.407 |
| 206 | IL4_3.125 nM | 9.49E+07 | 1.35E−04 | 51 | 1.43E−12 | 1.23 |

TABLE 16
Biacore measurement of protein variant (9) immobilized
with the aid of the human antibody capture kit.

| | | | | Rmax | | |
|---|---|---|---|---|---|---|
| RU 2nd Ab | Analyte | ka (1/Ms) | kd (1/s) | (RU) | KD (M) | Chi2 |
| 226 | IL13_25 nM | 3.18E+06 | 1.16E−04 | 28 | 3.64E−11 | 0.324 |
| 207 | IL4_3.125 nM | 1.77E+07 | 1.08E−04 | 39 | 6.09E−12 | 0.674 |

TABLE 17
Biacore measurement of protein variant (10) immobilized
with the aid of the human antibody capture kit.

| | | | | Rmax | | |
|---|---|---|---|---|---|---|
| RU 2nd Ab | Analyte | ka (1/Ms) | kd (1/s) | (RU) | KD (M) | Chi2 |
| 247 | IL13_25 nM | 2.49E+06 | 8.26E−05 | 25 | 3.31E−11 | 0.521 |
| 225 | IL4_3.125 nM | 9.19E+08 | 2.04E−04 | 32 | 2.22E−13 | 0.84 |

TABLE 18
Biacore measurement of protein variant (11) immobilized
with the aid of the human antibody capture kit.

| | | | | Rmax | | |
|---|---|---|---|---|---|---|
| RU 2nd Ab | Analyte | ka (1/Ms) | kd (1/s) | (RU) | KD (M) | Chi2 |
| 286 | IL4 0.39 nM | 6.84E+07 | 8.91E−05 | 18 | 1.30E−12 | 0.251 |
| 290 | IL13 25 nM | 1.69E+06 | 3.31E−05 | 31 | 1.96E−11 | 0.589 |

TABLE 19
Biacore measurement of protein variant (12) immobilized
with the aid of the human antibody capture kit.

| | | | | Rmax | | |
|---|---|---|---|---|---|---|
| RU 2nd Ab | Analyte | ka (1/Ms) | kd (1/s) | (RU) | KD (M) | Chi2 |
| 300 | IL4 0.39 nM | 2.42E+11 | 2.23E−01 | 22 | 9.22E−13 | 0.256 |
| 301 | IL13 25 nM | 1.59E+06 | 1.31E−05 | 29 | 8.24E−12 | 0.320 |

TABLE 20
Biacore measurement of protein variant (13) immobilized with the
aid of the human antibody capture kit. (anti-IL13, anti-IL4 CODV)

| | | | | Rmax | | |
|---|---|---|---|---|---|---|
| RU 2nd Ab | Analyte | ka (1/Ms) | kd (1/s) | (RU) | KD (M) | Chi2 |
| 205 | IL13_25 nM | 3.86E+06 | 9.45E−05 | 27 | 2.45E−11 | 0.519 |
| 189 | IL4_3.125 nM | 5.03E+08 | 1.37E−03 | 29 | 2.72E−12 | 0.576 |

1. A protein complex comprising at least two polypeptide chains A (PCA) and B (PCB), wherein PCA comprises a heterodimerization domain A (HDA) and PCB comprises a heterodimerization domain B (HDB) that bind to each other and wherein one heterodimerization domain comprises or consists of two N-terminal ß-strands of an immunoglobulin (Ig) domain (N-ß) and the other heterodimerization domain comprises or consists of two C-terminal ß-strands of an Ig domain (C-ß).

2. The protein complex according to claim 1, wherein
a. N-ß comprises a continuous amino acid sequence of an Ig domain comprising at least ß-strand b and c and
b. C-ß comprises a continuous amino acid sequence of an Ig domain comprising at least ß-strand e and f.

3. The protein complex according to claim 1 or 2, wherein the Ig domains of N-ß and C-ß are independently selected from an IgA, IgD, IgE, IgG, IgG1, IgG2, IgG3, or IgG4 heavy chain constant domain 2 (CH2), and an IgM or IgE heavy chain constant domain 3 (CH3), and are optionally selected from the same CH2 or CH3.

4. The protein complex according to claim 1, wherein HDA and HDB (i) non-covalently or (ii) non-covalently and covalently bind to each other.

5. The protein complex according to claim 3, wherein N-ß comprises or consists of a continuous amino acid sequence of a CH2 or CH3 domain comprising or consisting of ß-strand a to ß-strand c and C-ß comprises or consists of a continuous amino acid sequence of a CH2 or CH3 domain comprising or consisting of ß-strand e to ß-strand g.

6. The protein complex according to claim 1, wherein the N-ß and C-ß each comprise a non-naturally occurring Cys residue and wherein the Cys residues replace amino acids in the folded N-ß and C-ß, respectively, that naturally have a distance of between 3 to 7.5 Å between their Cα-atoms.

7. The protein complex according to claim 1, wherein HDA comprises or consists of:
PSVFLFPPKPKDTLMISRTPEVTCVVVDVSX₁EDPEVX₂FX₃WYVDGVEVHN (SEQ ID NO: 1),
wherein X₁ is H or Q, X₂ is K or Q and X₃ is N or K; or a variant thereof with an amino acid sequence having at least 80%, 85%, 90%, 95%, 96%, 97%, 98% identity to SEQ ID NO: 1; and/or HDB comprises or consists of:
NSTX₄RVVSVLTVX₅HQDWLNGKEYKCKVSNKX₆LPX₇X₈IEKTI (SEQ ID NO: 2),
wherein X₄ is Y or F; X₅ is L or V; X₆ is A or G; X₇ is K or Q; X₈ is N or K; or a variant thereof with an amino acid sequence with at least 80%, 85%, 90%, 95%, 96%, 97%, 98% identity to SEQ ID NO: 2, wherein SEQ ID NO: 1 or its variant can heterodimerize with SEQ ID NO: 2 or its variant.

8. The protein complex according to claim 1, wherein the complex comprises one or more antigen binding sites within the PCA and/or the PCB, wherein each of the one or more antigen binding sites is formed by a pair of two domains, wherein one is comprised in the PCA and the other is comprised in the PCB, and wherein the antigen binding site(s) are located N- and/or C-terminally of the HDA or HDB.

9. The protein complex according to claim 1, wherein the PCA and/or the PCB comprises one or more further homo and/or heterodimerization domain C (HDC), wherein the homodimerization domain is selected from the group consisting of a CH3 domain, a CH2-CH3 domain, or a domain where homodimerization is mediated by a an Ig-like fold, a rossmann- or rossmann-like alpha-beta-alpha sandwich fold, an alpha-sandwich fold, a continuous-beta-sheet fold, a beta-sandwich fold, a mixed beta-sheet fold, a 2-helix orientation, an antiparallel alpha-helix-orientation, a parallel alpha-helix orientation, a 4-helix bundle motif, a leucine zipper and a coiled-coil domain and the heterodimerization domain is selected from the group consisting of a knob-into-hole CH3 domain, a knob-into-hole CH2-CH3 domain, a Fc-domain with introduced mutations to force heterodimerization (e.g. charged mutations), a domain of a pair of interchanged domains (such as Fc-one/kappa heterodimerization domain, CL and CH domains), an Ig-like fold with introduced mutations to force heterodimerization, or a domain mediating heterodimerization containing a rossmann- or rossmann-like alpha-beta-alpha sandwich fold, an alpha-sandwich fold, a continuous-beta-sheet fold, a beta-sandwich fold, a mixed beta-sheet fold, a 2-helix orientation, an antiparallel alpha-helix-orientation, a parallel alpha-helix orientation, a 4-helix bundle motif, a leucine zipper and a coiled-coil domain; wherein the antigen binding site(s) are located N- and/or C-terminally of the HDC.

10. The protein complex according to claim 8, wherein PCA and PCB comprise from N- to C-terminus the following elements:
(i) PCA: V2-L1-HDA, and PCB: V2-L2-HDB;
(ii) PCA: V1-L3-HDA-L4-V2, and PCB: V1-L1-HDB-L2-V2;
(iii) PCA: V1-L1-V2-L2-HDA, and PCB: V2-L3-V1-L4-HDB;
(iv) PCA: V1-L3-V2-L5-CL-L4-HDA, and PCB: V1-L1-V2-L6-CH1-L2-HDB;
wherein L5, CL, L6 and CH1 may be present or absent; or
(v) PCA: V1-L4-V2-L5-CL-L6-HDA, and PCB: V2-L1-V1-L2-CH1-L3-HDB;
wherein L5, CL, L2 and CH1 may be present or absent;
wherein each pair of V1, V2, V3, and V4 comprises a variable domain of a heavy chain and a variable domain of a light chain or a variable domain of an alpha chain and a variable domain of a beta chain and forms an antigen binding site, wherein L1 to L6 are peptide linkers, and wherein the PCA and/or the PCB optionally further comprises a HDC.

11. The protein complex according to claim 10, wherein the PCA and/or the PCB comprises a first HDC, and wherein the complex comprises one or more additional polypeptides comprising one or more antigen binding sites and a second HDC that is covalently or non-covalently bound to the first HDC.

12. One or more polynucleotides encoding one or more polypeptides of the protein complex according to claim 1.

13. One or more expression vectors comprising the one or more polynucleotides according to claim 12.

14. A cell comprising the one or more polynucleotides of claim 12 or one or more expression vectors comprising said one or more polynucleotides.

15. A pharmaceutical composition comprising a pharmaceutically acceptable carrier and the protein complex according to claim 1, the one or more polynucleotides encoding one or more polypeptides of said protein complex, or one or more expression vectors comprising said one or more polynucleotides.
